(19) United States
(12) Patent Application Publication
(10) Pub. No.: US 2014/0296844 A1
(43) Pub. Date: Oct. 2, 2014

(54) DUAL BRACKETED ENERGY DELIVERY PROBE AND METHOD OF USE

(71) Applicant: ANGIODYNAMICS, INC., Latham, NY (US)

(72) Inventors: Moss Kevin, Tracy, CA (US); William M. Appling, Granville, NY (US)

(73) Assignee: ANGIODYNAMICS, INC., Latham, NY (US)

(21) Appl. No.: 14/304,316

(22) Filed: Jun. 13, 2014

Related U.S. Application Data
(63) Continuation of application No. 13/028,431, filed on Feb. 16, 2011.
(60) Provisional application No. 61/304,854, filed on Feb. 16, 2010.
(60) Provisional application No. 61/304,857, filed on Feb. 16, 2010.

(51) Int. Cl. A61B 18/14; A61B 18/12

(52) U.S. Cl. CPC A61B 18/1487; A61B 18/1206; A61B 18/1477; A61B 2018/1467
USPC 606/34

(57) ABSTRACT

An energy delivery probe and method of using the energy delivery probe to treat a patient is provided herein. The energy delivery probe has at least one probe body having a longitudinal axis and at least a first trocar and a second trocar. At least a portion of each trocar is disposed with the at least one probe body. The distance between the first trocar and the second trocar is adjustable between a first position and a second position. Each of the deployed electrodes has an energy delivery surface of a sufficient size to create a volumetric ablation zone between the deployed electrodes. The energy delivery probe is connected to an energy source. At least one cable couples the energy delivery probe to the energy source.

### CROSS-REFERENCE TO RELATED APPLICATIONS

This application claims priority to U.S. Provisional Application No. 61/304,854, filed Feb. 16, 2010; U.S. Provisional Application No. 61/304,857, filed Feb. 16, 2010; and U.S. Nonprovisional application Ser. No. 13/028,431, filed Feb. 16, 2011; all of which are incorporated by reference herein in their entireties.

### TECHNICAL FIELD

The present invention relates to an energy delivery probe and method of treatment using the energy delivery probe.

### BACKGROUND OF THE INVENTION

Irreversible electroporation (IRE) is a non-thermal, minimally invasive surgical technique to ablate undesirable tissue, for example, tumor tissue. The technique is easy to apply, can be monitored and controlled, is not affected by local blood flow, and does not require the use of adjuvant drugs. The minimally invasive procedure involves placing needle-like electrodes into or around a targeted tissue area to deliver a series of short and intense electric pulses that induce structural changes in the cell membranes that promote cell death.

Among the problems associated with current IRE procedures is that during a single IRE ablation, a practitioner may need to place up to six separate needles parallel to each other with uniform spacing between each needle in order to perform a single ablation treatment. However, when using any of the single needle products currently commercially available for Irreversible Electroporation (IRE) ablations, it can be difficult and time consuming for practitioners to place multiple needles into a patient during treatment, while keeping each of the needles parallel to each other with uniform spacing between each needle before and during treatment. Current single bracket electrode designs can be difficult to insert and deploy while maintaining the trocars in a parallel position. Current single needle IRE bipolar devices are capable of creating maximum ablations of about 1.5 cm in diameter or treating tumors of about 0.5 cm³ in volume. Given this ablation size, such devices can be limiting.

Another technique for ablating a desired target tissue is radiofrequency ablation (RFA). This procedure involves using an imaging guidance system such as ultrasound (US), computed tomography (CT), or magnetic resonance (MR). During this procedure, the doctor places a probe directly into a target tissue area, such as a tumor. Using an energy source, such as, but not limited to, a radiofrequency generator, a physician or other practitioner can then deliver a carefully-controlled amount of energy to flow through the electrodes into the tissue which causes the tissue to heat up. The heating is sustained for a predetermined length of time, usually just a few minutes, which kills and destroys the target tissue. RFA procedures can be percutaneously or laparoscopically performed.

The majority of the commercially available RFA products on the market today are of a monopolar design, meaning that they each require the use of ground pads to be placed on a patient in order to complete an electrical circuit during treatment and to allow the radio frequency (RF) energy to be conducted back to an RF generator. The correct placement of these pads is critical for the proper operation of the RFA device, as well as protecting the patient from unwanted burns caused by return energy being directed to the wrong location. In addition, with the separate return path that is conducted through a patient's body back to the ground pads, there can be a large amount of energy loss due to the resistance of body tissue, thereby limiting the amount of actual energy delivered to a monopolar device. Because only limited energy can be delivered safely to the RFA device, such RFA procedures take longer and have a risk of unwanted burns around the return pads.

There exists a need in the art for an improved probe and method of using such a probe that will allow for improved IRE and RF ablations that can function as bipolar devices, allow for larger ablations, and provide the ability to easily maintain the electrodes in a parallel position before, during, and after an ablation. An electrode probe and method has not yet been proposed that would solve the problems described above, thereby avoiding many of the negative side effects of the current devices described above.

It is a purpose of the invention described herein to provide a dual bracketed probe that can be used for either IRE or RF ablations.

It is a purpose of this invention to provide a dual bracketed probe that is capable of producing bipolar energy that enables ablations to occur in a shorter time period than is currently seen with commercially available devices.

It is a purpose of this invention to provide a dual bracketed probe having electrodes that can be deployed parallel to each other into a target tissue in a patient that can remain uniformly spaced before, during, and after insertion of the probe into a target tissue and treatment of a patient.

It is also a purpose of this invention to provide a dual bracketed probe that has an electrode or trocar spacing design that is adjustable, but yet will allow the electrodes or trocars to remain parallel to each other throughout a complete adjustment range.

It is a purpose of this invention to provide a dual bracketed probe that can be used to produce IRE or RF ablation zones that are at least equivalent to or greater than current typical IRE or RF ablation zones that are possible when using six individual single needles placed in a parallel position, as found in current commercially available bipolar IRE devices, in order to make an equivalent ablation.

It is a purpose of this invention to provide a dual bracketed probe that has an electrode spacing that can be adjusted to accommodate multiple sized ablations and to produce larger ablations than are typically feasible using one single probe device, depending on the size of the target tissue to be ablated.

It is a purpose of the invention to provide a dual bracketed probe that can be placed individually as two separate electrodes or one dual electrode design that has adjustable, parallel electrodes.

Various other objectives and advantages of the present invention will become apparent to those skilled in the art as more detailed description is set forth below. Without limiting the scope of the invention, a brief summary of some of the claimed embodiments of the invention is set forth below. Additional details of the summarized embodiments of the invention and/or additional embodiments of the invention can be found in the Detailed Description of the Invention.

### SUMMARY

An energy delivery probe for treating a patient is provided herein. The energy delivery probe has at least one probe body having a longitudinal axis, at least a first trocar and a second trocar. A portion of each trocar is disposed with the at least one probe body. The trocars each have a proximal portion and a distal portion. Each of the distal portions is capable of piercing tissue, and at least one hollow lumen extending along a longitudinal axis. The distance between the first trocar and the second trocar is adjustable between a first position and a second position.

The first trocar and the second trocar of the energy delivery probe can be defined in a substantially parallel relationship relative to each other. The energy delivery probe can also include a plurality of electrode arrays, each electrode having a proximal portion and a distal portion. The plurality of electrodes are at least partially positioned within the trocars and adapted to be deployed radially away from probe body and into tissue of a patient. The plurality of electrodes is adapted to receive electrical treatment energy from an energy source.

A method of treating a patient using an energy delivery probe is provided herein. The method comprises includes identifying a target tissue and providing at least one energy delivery probe device. The energy delivery probe includes at least one probe body, at least a first trocar and a second trocar having a longitudinal axis, and a plurality of electrode arrays. The trocars are substantially parallel in relation to each other, and the electrode arrays are defined within a portion of the trocars. The method includes inserting the first trocar and the second trocar into tissue such that the target tissue is substantially positioned between the first and second trocars; deploying the plurality of electrode arrays radially away from the longitudinal axis of the trocars into the tissue; and delivering energy to the target tissue to ablate the tissue, thereby forming a first ablation zone.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 illustrates a perspective view of a first embodiment of an energy delivery probe device in a deployed state.

FIG. 2A illustrates a plan view of the energy delivery probe device illustrated in FIG. 1.

FIG. 2B illustrates an enlarged side view of the distal end of the energy delivery probe device illustrated in FIGS. 1 and 2A.

FIG. 3A illustrates an enlarged perspective view of the distal end of the probe of FIGS. 1-2B in an undeployed state.

FIG. 3B illustrates an enlarged side view of the distal end of the energy delivery probe of FIG. 3A.

FIG. 3C illustrates an enlarged side view of an alternative embodiment of the distal end of the energy delivery probe of FIG. 3A.

FIG. 4A illustrates an enlarged side view of an alternative embodiment of the distal end of the energy delivery probe of FIG. 1.

FIG. 4B illustrates an enlarged side view of an alternative embodiment of the distal end of the energy delivery probe of FIG. 1.

FIG. 4C illustrates an enlarged side view of an alternative embodiment of the distal end of the energy delivery probe of FIG. 1.

FIG. 4D illustrates an enlarged side view of an alternative embodiment of the distal end of the energy delivery probe of FIG. 1.

FIG. 4E illustrates an enlarged side view of an alternative embodiment of the distal end of the energy delivery probe of FIG. 1.

FIG. 5A illustrates a perspective view of another embodiment of the energy delivery probe.

FIG. 5B illustrates a perspective view of the spacer of FIG. 5A.

FIG. 6 illustrates a perspective view of another embodiment of the energy delivery probe with a pre-assembled spacer.

FIGS. 7A and 7B illustrate top views of the separable components of the energy delivery probe of FIG. 6.

FIG. 7C illustrates a perspective view of the energy delivery probe of FIGS. 7A and 7B.

FIG. 8 illustrates a perspective view of another embodiment of the energy delivery probe.

FIG. 9A illustrates a perspective view of the distal portion of the energy delivery probe in which the trocars are positioned a maximum distance from each other.

FIG. 9B illustrates a front end view of the energy delivery probe of FIG. 9A.

FIG. 9C illustrates a top cutaway view of the energy delivery probe of FIG. 9A.

FIG. 10A illustrates a perspective view of the distal portion of the energy delivery probe of FIG. 8 wherein the trocars are positioned at a parallel minimum distance from each other.

FIG. 10B illustrates a front end view of the energy delivery probe illustrated in FIG. 10A.

FIG. 10C illustrates a top cutaway view of the distal portion of the energy delivery probe of FIG. 10A.

FIG. 11A is a perspective view of a different partial embodiment of the energy delivery probe.

FIG. 11B is an enlarged perspective view of the distal portion of the energy delivery probe of FIG. 11A.

FIG. 11C is a front end view of the distal portion of the probe of FIG. 11A.

FIG. 12 is a perspective view of a portion of the distal end of an alternative embodiment of the energy delivery probe of FIG. 11A.

FIG. 13A illustrates a method of using an energy delivery probe such as illustrated in FIG. 5 to ablate a target tissue.

FIG. 13B illustrates a front end view of the energy delivery probe of FIG. 13A in relationship to a target tissue.

FIG. 14 illustrates a method of using an energy delivery probe such as illustrated in FIGS. 8 through 10C to ablate a target tissue.

FIG. 15A illustrates one embodiment of an energy delivery pattern using an energy delivery probe.

FIG. 15B illustrates another embodiment of an energy delivery pattern using an energy delivery probe.

FIG. 15C illustrates another embodiment of an energy delivery pattern using an energy delivery probe.

FIG. 16 illustrates a predicted ablation zone using the distal electrode configuration of the energy delivery probe illustrated in FIG. 5.

FIG. 17 illustrates another predicted ablation zone using the distal electrode configuration of the energy delivery probe illustrated in FIG. 5.

FIG. 18 illustrates a photograph of ablation zones of several pig liver tissues following an ablation.

FIG. 19 illustrates a photograph of an ablation zone in a partial section of one of the pig liver tissues illustrated in FIG. 18 following an ablation.

FIG. 20 illustrates a photograph of an ablation zone in a partial section of pig liver tissue illustrated in FIG. 19 following an ablation.

FIG. 21 illustrates a photograph of ablation zones of several pig liver tissues following an ablation.

FIG. 22 illustrates a photograph of an ablation zone in a partial section of one of the pig liver tissues illustrated in FIG. 21 following an ablation.

FIG. 23 illustrates a photograph of an ablation zone in a partial section of pig liver tissue illustrated in FIG. 19 following an ablation.

FIG. 24 illustrates a photograph of ablation zones of several pig liver tissues following an ablation.

FIG. 25 illustrates a photograph of an ablation zone in a partial section of one of the pig liver tissues illustrated in FIG. 24 following an ablation.

FIG. 26 illustrates a photograph of an ablation zone in a partial section of pig liver tissue illustrated in FIG. 25 following an ablation.

### DETAILED DESCRIPTION OF THE INVENTION

The present invention can be understood more readily by reference to the following detailed description and the examples included therein and to the Figures and their previous and following description. The drawings, which are not necessarily to scale, depict selected preferred embodiments and are not intended to limit the scope of the invention. The detailed description illustrates by way of example, not by way of limitation, the principles of the invention.

The skilled artisan will readily appreciate that the devices and methods described herein are merely exemplary and that variations can be made without departing from the spirit and scope of the invention. It is also to be understood that the terminology used herein is for the purpose of describing particular embodiments only and is not intended to be limiting.

Ranges can be expressed herein as from “about” to one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. As used herein, the words “proximal” and “distal” refer to directions away from and closer to, respectively, the insertion tip of the probe in the probe. The terminology includes the words above specifically mentioned, derivatives thereof, and words of similar import.

Other than in the operating examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values can be used.

“Formed from” and “formed of” denote open claim language. As such, it is intended that a member “formed from” or “formed of” a list of recited components and/or materials be a member comprising at least these recited components and/or materials, and can further include other non-recited components and/or materials.

Examples provided herein, including those following “such as” and “e.g.,” are considered as illustrative only of various aspects and features of the present disclosure and embodiments thereof, without limiting the scope of any of the referenced terms or phrases either within the context or outside the context of such descriptions. Any suitable equivalents, alternatives, and modifications thereof (including materials, substances, constructions, compositions, formulations, means, methods, conditions, etc.) known and/or available to one skilled in the art can be used or carried out in place of or in combination with those disclosed herein, and are considered to fall within the scope of the present disclosure. Throughout the present disclosure in its entirety, any and all of the one, two, or more features and aspects disclosed herein, explicitly or implicitly, following terms “example”, “examples”, “such as”, “e.g.”, and the likes thereof may be practiced in any combinations of two, three, or more thereof (including their equivalents, alternatives, and modifications), whenever and wherever appropriate as understood by one of ordinary skill in the art. Some of these examples are themselves sufficient for practice singly (including their equivalents, alternatives, and modifications) without being combined with any other features, as understood by one of ordinary skill in the art. Therefore, specific details disclosed herein are not to be interpreted as limiting, but merely as a basis for the claims and as a representative basis for teaching one skilled in the art to variously employ aspects and features of the present disclosure in virtually any appropriate manner.

As used herein, “substantially”, “generally”, and other words of degree are relative modifiers intended to indicate permissible variation from the characteristic so modified. It is not intended to be limited to the absolute value or characteristic which it modifies, but rather possessing more of the physical or functional characteristic than its opposite, and preferably, approaching or approximating such a physical or functional characteristic. “Optional” or “optionally” means that the subsequently described element, event or circumstance can or cannot occur, and that the description includes instances where said element, event or circumstance occurs and instances where it does not. The term “ablation” is used herein to refer to either irreversible electroporation (IRE) ablations or radiofrequency ablation (RFA) ablations or both. “IRE ablation device” is used herein to refer to any of the devices described herein that can be used for IRE ablations. “RFA devices” can be used herein to refer to any of the devices described herein that can be used for RF ablations. All dimensions herein are exemplary, and one of ordinary skill in the art will recognize that other dimensions possible.

Referring now in detail to the drawings, in which like reference numerals indicate like parts or elements throughout the several views, in various embodiments, presented herein is an exemplary ablation device, such as a dialysis ablation device, and a method of treatment using the dialysis probe in a human lung.

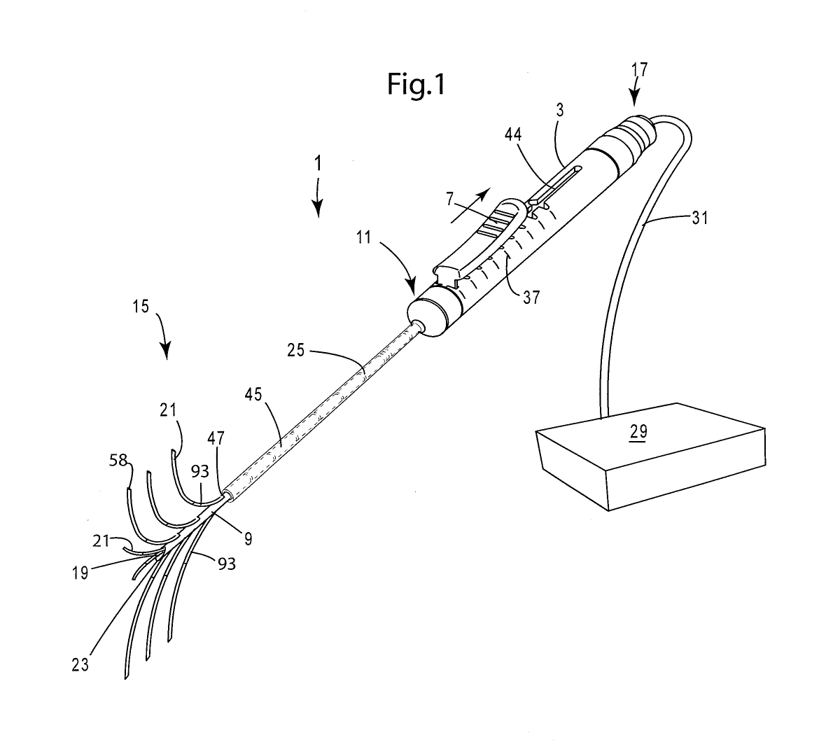

FIGS. 1 through 3C illustrate one exemplary embodiment of an energy delivery probe 1 for use in treating a patient. The probe can be an RF ablation probe or an IRE ablation probe. The probe 1 has a proximal end 17, a distal end 15 and a longitudinal axis. At least a portion of the proximal end 17 of the probe 1 can be configured to be positioned outside of a human body. At least a portion of the distal end 15 of the probe 1 can be configured to be inserted into at least a portion of a human body, such as, but not limited to, a target tissue.

The probe 1 further comprises a probe body. The probe body comprises a handle 3 that can be positioned at the proximal end 17 of the probe 1. The probe body can be substantially fixed in relation to the first trocar 9 and the second trocar 9. The proximal end 17 of the probe and the proximal end of the handle 3 are referred to herein interchangeably. The handle 3 has a distal end 11, an outer surface, and an inner cavity. The probe 1 can be operatively coupled at the proximal end of the handle 17 to a power source 29 by at least one cable 31. A portion of the cable 31 is positioned within at least a portion of the handle 3, such that the at least one cable 31 is adjacent to the proximal end of the probe 1 and extends outwardly from the proximal end 17 of the handle 3.

The power source can be, but is not limited to, an RF source, electrical energy source, microwave source, short wave source, laser source and the like. In one aspect, the energy source 29 can be a generator 29. The generator 29 is configured for supplying energy to the probe 1 in a controlled manner. The energy delivery source can be capable of delivering energy that selected from the group comprising: radiofrequency (RF) energy and electrical energy. Such generators are commercially available from AngioDynamics, Inc. (Latham, N.Y.) and can include, but are not limited to, AngioDynamics' RITA® 1500X RF generator or NanoKnife® generator.

The probe 1 further comprises at least one elongate body. The elongate body can be a trocar 9. The trocar 9 comprises at least one electrode 21. The trocar 9 has a proximal end and a distal end. At least a portion of the trocar 9 can function like an electrode. Therefore, the terms trocar 9 and electrode 9 may be used interchangeably herein. At least a portion of the trocar 9 can be positioned within the cavity of the handle 3 and is operatively coupled to at least a portion of the handle 3. The at least one trocar 9 and the handle 3 extend along the longitudinal axis of the probe 1. The handle 3 comprises at least one slot 44. The slot 44 is defined within the outer surface of the handle 3 and extends along the longitudinal axis of the probe. The slot 44 further comprises a plurality of grooves 85 that are positioned at a substantially right angle to the longitudinal axis of the probe.

The probe further comprises a first slide member 7 that is slideably disposed on the handle 3. At least a portion of the slide member 7 is received within slot 44. The slide member 7 can be slideably actuated in a proximal or a distal direction along the longitudinal axis of the probe 1 such that at least a portion of the slide member 7 can be received and locked into place in a single groove 85. Each groove 85 corresponds with an index marking 37. Each marking 37 corresponds with an electrode deployment size and can be used to indicate to a user the required depth of electrode deployment from trocar 9 needed for 2, 3, and 4 cm diameter tissue ablations, for example. At least a portion of the slide member 7 is operatively coupled to a portion of at least one electrode array 21, described below. As illustrated in FIG. 1, the slide member 7 can be distally actuated to deploy the arrays 21 or proximally actuated, as indicated by the arrow, to retract the arrays 21 with a portion of the trocar 9.

The trocar 9 has a proximal end that is positioned within at least a portion of the handle 3 and a distal end 15. A portion of each trocar 9, 90 can be disposed with the at least one probe body. The distal end 15 of the trocar 9 and the distal end of the probe 1 are used interchangeably herein. The trocar 9 extends distally from the handle 3 to a distal tip 23. The distal tip 23 can be sharp enough so that it is capable of piercing tissue. The trocar 9 can have at least one lumen 19 that extends along the longitudinal axis of the probe 1. If the probe 1 is an RF probe, the trocar 9 can be comprised of stainless steel or Inconel. If the probe 1 is an IRE probe, the trocar 9 can be comprised of a non-conductive material such as, but not limited to, polyimide or PEEK (polyether ether ketone). In one exemplary embodiment, the trocar 9 can be from about 13 gauge to about 15 gauge (1.828 mm to 1.449 mm) in size, depending on the desired treatment or a patient's anatomy. The trocar 9 can have a uniform diameter throughout its longitudinal length. The working length of the trocar 9 can be between about 10 cm and about 25 cm. The working length of the trocar is defined from a point just distal of the distal end of the handle 3 to the distal tip 23 of the trocar, depending on the size of the target tissue to be ablated and a patient's anatomy.

The trocar 9 can comprise at least one index marker, such as, but not limited to, at least one depth marking 25, positioned along the outer surface of the trocar 9. The depth markings 25 can be fixed in place and equi-distantly positioned from one another. The depth markings 25 can be used to aid a practitioner in gauging the depth of deployment of the arrays 21 from the probe 1 and for determining a desired ablation depth.

In one embodiment, at least a portion of the trocar 9 can be rigid for IRE probes, but flexible or semi-flexible for RF probes. The rigid body and sharp tips of the trocars 9, 90 can be useful for penetrating target tissues, especially large, hard tumors. In one aspect, as illustrated in FIGS. 1, 2B, and 3A, the trocar 9 can comprise a plurality of openings or side ports 47 defined therein the outer wall of the trocar 9. The trocar 9 can have between about 1 and 8 openings 47. The plurality of openings or side ports 47 can be positioned in an equi-distant arrangement within the external wall of the trocar 9 such that each opening or side port 47 is in communication with the lumen 19 of the trocar 9. The plurality of openings or side ports 47 are defined in the outer surface of the trocar 9 and are configured to allow the electrode arrays 21 to de deployed through the openings.

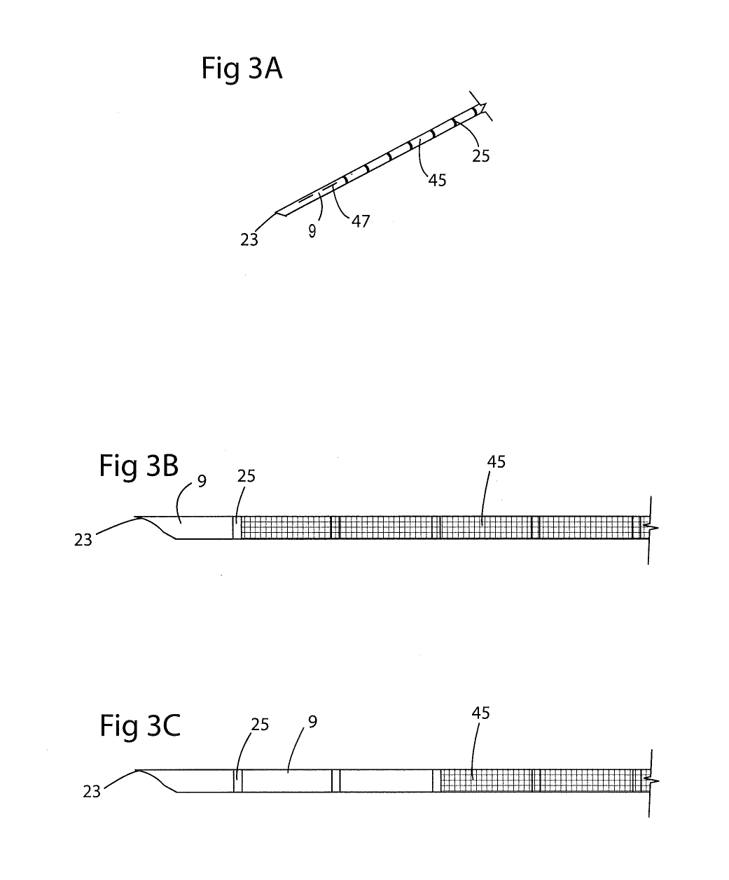

As illustrated in FIGS. 1 through 3C, at least a portion of the outer surface of the trocar 9 can be completely electrically insulated from the arrays 21 by an insulative sleeve 45. In one embodiment, insulation sleeve 45 can comprise a polyamide material. The insulation sleeve 45 can be semi-rigid. The insulative sleeve 45 can extend from the proximal end of the trocar 9 to within about 0.25 to about 0.5 inches from the openings 47. RF probes 1 may optionally include an insulative sleeve 45. The insulation sleeve 45 may be positioned in a surrounding relationship around at least a portion of an exterior of the trocar 9. Particularly, the insulative sleeve 45 can be coaxially positioned around at least a portion of the trocar 9 and can be permanently fixed in place. A distal end of the insulation sleeve 45 at the distal end of the trocar 9 can be removed. This creates an energy delivery surface at the trocar's distal end. The trocar then becomes at least partially an electrode. One of ordinary skill in the art will recognize that the insulation sleeve 45 can be adjusted along the length of the trocar 9 to any desired position, as illustrated in FIGS. 3B and 3C. All or some portion of the insulation sleeves 45 may be adjustably positioned so that the length of an energy delivery surface of a trocar 9 can be varied. The thickness of the insulation 45 can vary, depending on whether the probe is an IRE probe or an RF probe. The insulation thickness may be varied because the operating voltage and currents of IRE and RF devices can be significantly different.

In one aspect, as illustrated in FIGS. 1 through 2B and 4A through 4E, the probe 1 can further comprise at least one electrode array 21. In one aspect, the trocar 9 is coupled to a plurality of electrode arrays 21. In other embodiments, the probe 1 can have any suitable number of electrode arrays 21. The electrode arrays 21 can be slidably disposed within a portion of the lumen 19 of the elongate trocar 9. The electrode arrays 21 can be configured for passage through the plurality of openings 47 that are positioned in the outer wall of the trocar 9. The trocar 9 can comprise between about 1 and about 8 arrays 21.

In one aspect, the arrays 21 can be comprised of a shape memory material, such as, but not limited to, Nitinol, stainless steel, and other suitable materials. The electrode arrays can have a pre-curved, non-linear shape that is biased to assume a desired configuration when advanced into a target tissue or region of tissue. At least a part of a distal portion of each deployed electrode array 21, 210 is constructed to be structurally less rigid than the trocar 9. Structural rigidity is determined by, (i) choosing different materials for trocar 9 and distal end of the electrode arrays 21 or some greater length of electrode arrays 21, (ii) using the same material but having less of it for the electrode array 21 or the material is not as thick as trocar 9, or (iii) including another material in trocar 9 or an electrode array 21 to vary their structural rigidity. For purposes of this disclosure, structural rigidity is defined as the amount of deflection that an electrode arrays 21 has relative to its longitudinal axis. It will be appreciated that a given electrode 21 will have different levels of rigidity depending on its length. Electrode arrays 21 can be made of a variety of conductive materials, both metallic and non-metallic. One suitable material is type 304 stainless steel of hypodermic quality. In some applications, all or a portion of the electrode arrays 21 can be made of a shaped memory metal, such as NiTi (Raychem Corporation, Menlo Park, Calif.).

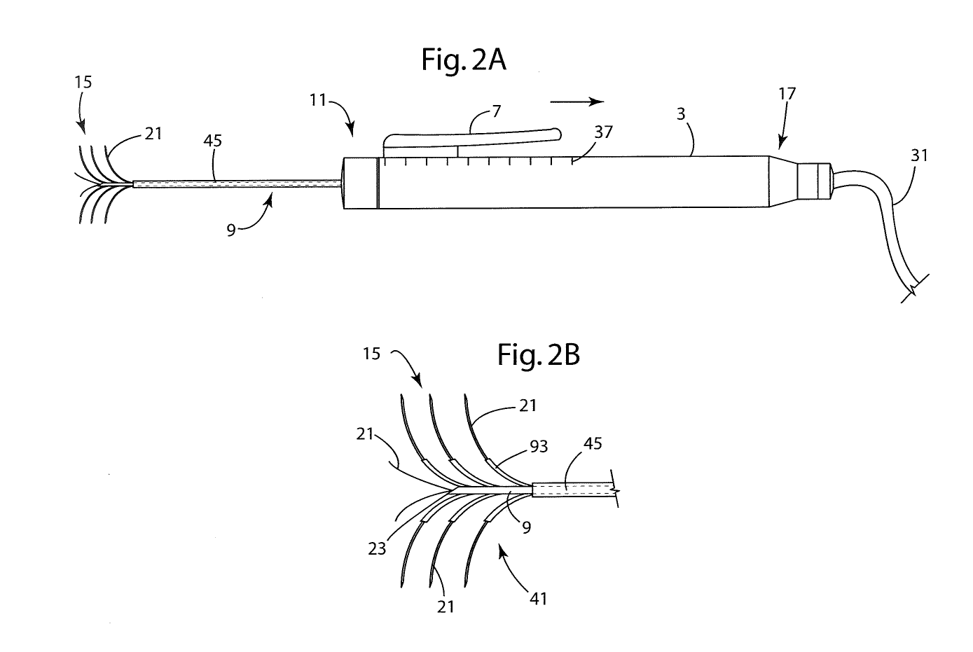

Each array 21 has a distal tip 58. Each tip 58 can be sharpened to facilitate the ability of the array tip 58 to penetrate tissue. The arrays 21 illustrated in FIGS. 1 through 2B, for example, can be about 17.5 mm in length. Although the electrode arrays 21 can have substantially identical lengths, in one aspect, each of the electrodes 21 can have different lengths. The lengths can be determined by the actual physical length of electrodes 21, the length of an electrode energy delivery surface, and the length of an electrode 21 that is not covered by an insulator 93. The actual length of an electrode 21 depends on the location of the selected tissue mass to be ablated, its distance from the skin, its accessibility as well as whether or not the physician chooses a percutaneous or other procedure. At least a part of each distal portion of a deployed electrode array 21 is configured to be deployable from the trocar lumen 19 at the tissue site with at least one radius of curvature. Each of the arrays 21 can be between about 0.016 and 0.020 inches in diameter. The arrays 21 can be solid, as illustrated, for IRE probes. Alternatively, for RF probes, the arrays 21 can be hollow and can comprise at least one thermocouple (not shown) in each array 21. The thermocouples can be used to measure the temperature at an end or outer boundary of a tissue ablation.

For IRE probes, the arrays 21 are at least partially coaxially surrounded by an insulation layer 93, as illustrated in FIGS. 1 through 2B. The additional insulation layer 93 can be fixed in place or it can be adjustable. The insulation layer 93 prevents the arrays 21 from shorting together inside of trocar 9. Each electrode array 21 is adapted to be deployed into target tissue through a corresponding deployed insulation sleeve 93. The arrays 21 can each have a pre-determined exposed length that provides an energy delivery surface at the distal end of each array 21 beyond each of the insulation sleeves 93. The energy delivery surface is capable of delivering energy to the tissue from energy source 29. The insulation sleeves 93 can also function as guide sleeves, as described in co-pending U.S. application Ser. No. 13/027,801, filed Feb. 15, 2011, incorporated herein by reference.

The collective size of the deployed electrodes arrays' 21 energy delivery surfaces is sufficient to create a volumetric ablation zone between the deployed electrodes when sufficient energy is delivered from the energy source to the ablation device. Volumetric ablation is defined as the creation of an ablation with a periphery formed between adjacent distal ends of the electrode arrays 21, 210. Unless the distal ends of the electrode arrays 21, 210 have insulation, then their entire length of extension is an energy delivery surface which delivers energy to the selected tissue mass. The length and size of each energy delivery surface can be variable. The lengths of the electrode arrays 21, 210 can be adjustable. Creation of different ablation geometries is dependent on the length of energy ablation delivery surfaces, the number of electrodes, the size of the delivery surfaces, the amount of power delivered to the electrodes 21, and the duration of time for power delivery to the electrodes.

Referring to FIGS. 1 through 2B, the arrays 21 of the probe 1 can be deployed from the lumen 19 of the trocar 9. To fully deploy the arrays, the slide member 7, which is operatively coupled to the arrays 21, can be slideably distally actuated along the handle 3. The array 21 configuration illustrated in the embodiment illustrated in FIGS. 1 through 2B comprises two sets of three arrays 21 positioned substantially equi-distantly from each other along a longitudinal axis. The electrode arrays 21 are deployed outwardly and laterally relative to the trocar's longitudinal axis from the trocar lumen 19 into a selected tissue mass along a radius of curvature from the openings or side ports 47 in the trocar 9. Each of the sets of three electrode arrays 21 are positioned on opposing sides of the trocar 9 in a mirrored configuration, for a total of six arrays 21. In other embodiments, the deployed electrode arrays 21 may have a non-mirrored orientation. Two additional electrode arrays 21 can be deployed distally from the distal end of the trocar lumen 19 of the trocar 9 along a radius of curvature, for a total of 8 arrays 21. In one aspect, all of the arrays 21 can be defined within a single plane that is parallel with the longitudinal axis of the trocar 9. The two most proximal arrays are the “proximal arrays”. The second set of arrays positioned distally of the first set of arrays is the “middle arrays”, and the remaining four electrodes are the “distal arrays”.

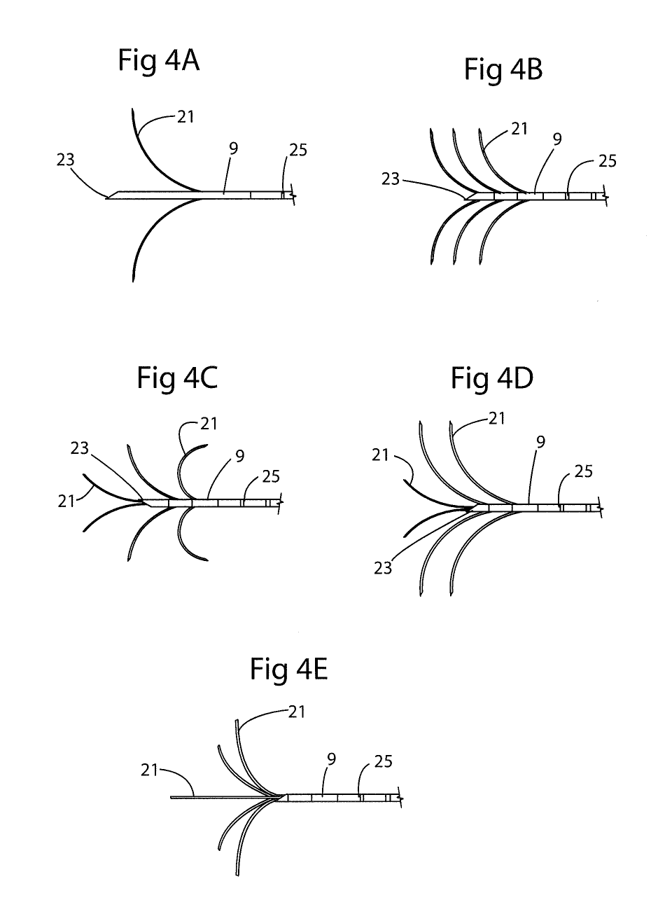

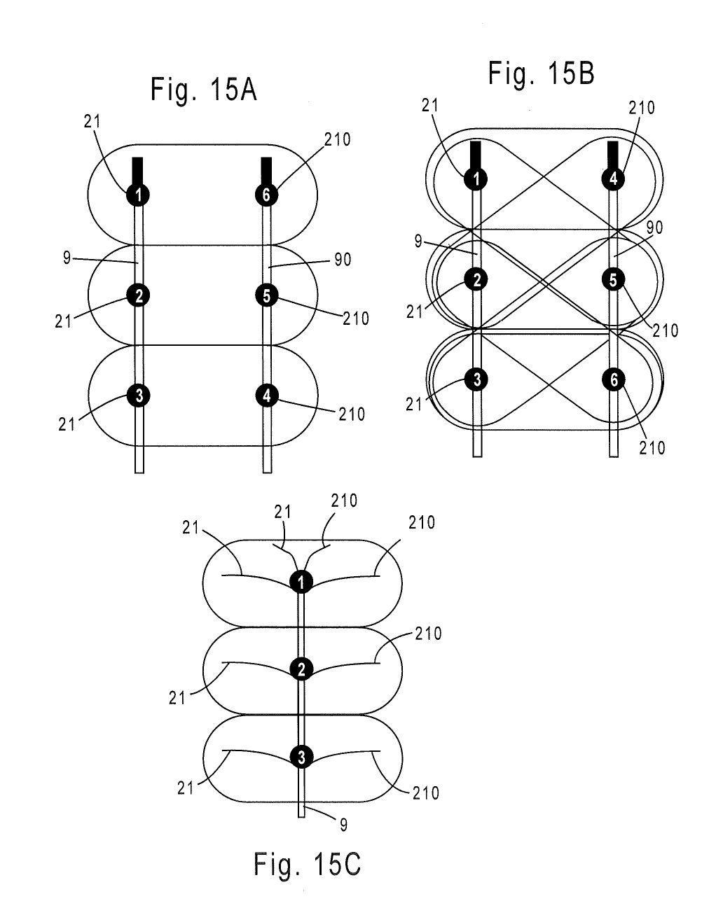

When deployed into tissue, the energy delivery probe 1 can have 1, 2, or 3 poles per electrode. In one exemplary embodiment, the probe 1 can have 3 poles per electrode or 6 poles total. For the probe 1 having the array configuration described in FIGS. 1 through 2B, the 2 proximal arrays function as a first pole, the 2 middle arrays function as a second pole, and the 4 distal arrays function as a third pole. This configuration is also illustrated in FIGS. 15A through 15C. The electrode arrays 21 can be spaced apart between about 38 mm and about 40 mm. The array tips 58 that extend outwardly from the trocar 9 can be spaced between about 18 mm and 20 mm from the trocar 9. Although one particular distal array embodiment is illustrated in FIGS. 1 through 2B, one of ordinary skill in the art will recognize that other array configurations 21 are contemplated as well, such as, but not limited to those illustrated in FIGS. 4A through 4E. Each of the arrays 21 is adapted to receive electrical treatment energy from energy source 29. During use, energy is delivered to the target tissue from energy source 29 through the energy delivery surfaces of the arrays 21 to the target tissue. In one aspect, the energy delivery probe 1 described herein can be configured to operate as a bipolar probe device. Such bipolar probes are described in U.S. patent application Ser. No. 12/437,843, filed May 8, 2009 (“Electroporation Probe and Method”), which application is incorporated herein by reference in its entirety.

Although not illustrated, in one aspect, any of the energy delivery devices described herein can optionally include at least one cooling mechanism. Such cooling mechanisms can comprise the infusion of one or more liquids through the lumen 19 of the trocar 9. The trocar lumen 19 may be coupled to an infusion medium source and deliver an infusion medium to the selected tissue site. A cooling element can be coupled to at least one of the electrodes. The cooling element can be a structure positioned in at least one of the electrodes and can include at least one channel configured to receive a cooling medium. The cooling medium can be recirculated through the channel. RF probes described herein can also optionally include temperature feedback circuitry.

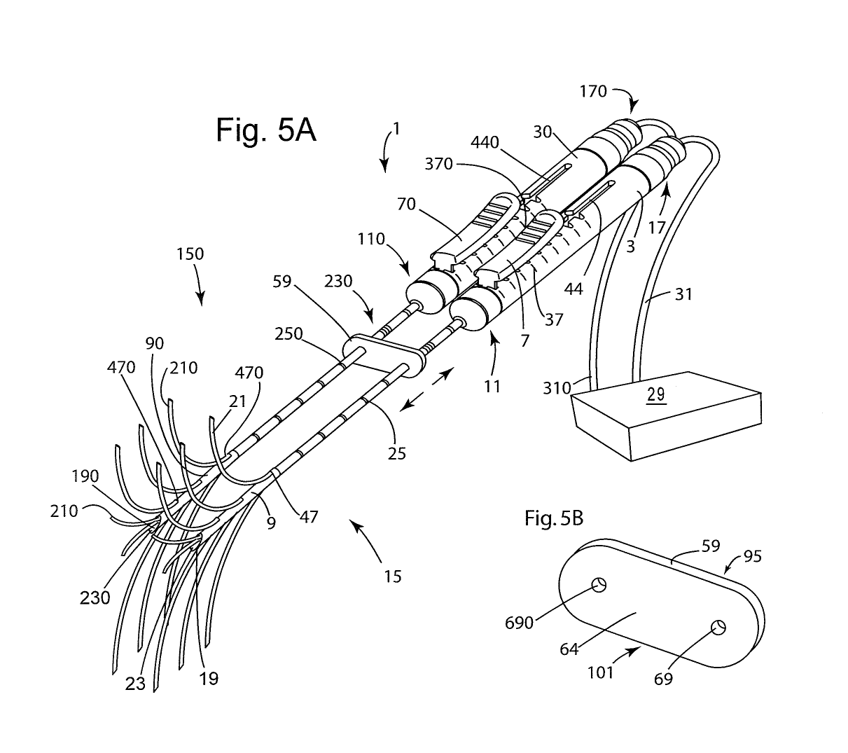

FIG. 5A illustrates a second embodiment of the probe 1. In this embodiment, the probe 1 can comprise two identical dual bracketed bipolar probes 1, 10, as described above and illustrated in FIGS. 1-2B. The dual bracketed probes 1, 10 are positioned substantially parallel relative to one another. Each of the trocars 9, 90 can be spaced apart at a desired distance from each other such that the ablation devices 1, 10, including the trocars 9, 90, remain parallel to one another at all times before, during, and after ablation. The trocars 9, 90 can be spaced at different distances from each other depending on whether the probes 1, 10 will be RF probes or IRE probes. In the embodiment illustrated in FIG. 5A, the trocars 9, 90 can be spaced about 20 mm apart, and the arrays 21 are positioned in a fully deployed state. The probes 1, 10 can comprise from about 1 to about 8 arrays 21 per trocar 9, or between about 2 and about 16 total electrode arrays 21. The bipolar dual bracketed probes 1, 10 described herein allow the creation of larger, faster ablations compared to current commercially available single RF or IRE ablation devices.

As illustrated in FIGS. 5A and 5B, a locking spacer 59 can be used to position and maintain the position of trocars 9, 90 such that they remain parallel to each other before, during, and after insertion and ablation treatment using the probes 1, 10. In one aspect, as illustrated in FIG. 5B, the locking spacer 59 can be a separate component that is capable of being axially slidably mounted onto at least a portion of the outer surface of the trocars 9, 90 for selectively positioning and retaining the pair of trocars 9, 90, and the probes 1, 10. The spacer 59 has a proximal end 95 and a distal end 101. The spacer 59 can be comprised of an ABS plastic material or a similar plastic material. The spacer 59 can have any desired shape or size, such as, but not limited to, square or rectangular. The spacer 59 can have rounded edges, as illustrated in FIG. 5B. In one aspect, the spacer 59 can be transparent so that the markers 25 on the trocar 9 can remain visible to a practitioner.

In one aspect, the spacer 59 can be between about 3 cm and 5 cm across the width of the trocars and between 1 and 3 cm in thickness along the longitudinal length of the trocars. The spacer 59 can have a body with an outer surface and at least two bores, a first bore 69 and a second bore 690. Each bore has an inner surface, and each bore 69, 690 is capable of receiving a portion of an outer surface of the first trocar 9 and the second trocar 90. The first and second bores 69, 690 can extend through the body of the spacer 59 such that they are in communication with the exterior of the spacer 59. The position of the bores 69, 690 within the spacer 59 can be adjusted to match a desired spacing between the trocars 9, 90. The bores 69, 690 are capable of receiving at least a portion of the outer surface of each of trocars 9, 90. Each of the bores 69, 690 of the spacer 59 can be equal to or slightly smaller in diameter than the outer diameter of the insulative sleeve 45 on the trocars 9, 90 in order to provide a sufficient interference fit between the outer surface of the insulative sleeve 45 and the inner surface of the bore 69, 690. Once the spacer 59 has been positioned along the trocars 9, 90, the interference fit between the outer surface of the insulative sleeve 45 and the inner surface of the bores 69, 690 can prevent the spacer 59 from sliding out of a desired position during insertion and use. Although not illustrated, in one alternative embodiment, the spacer 59 can further comprise a locking mechanism.

The spacer 59 can be slideably moveable or adjustable in either a proximal or a distal direction along the longitudinal length of the trocars 9, 90. In one exemplary embodiment, the spacer 59 can be configured to be received into small grooves (not shown) that can be positioned along the longitudinal length of the outer surface of the insulation sleeves 45, 450. Although the spacer 59 is illustrated in FIGS. 5A and 5B as a separate component used in conjunction with one particular embodiment of an probe 1, such as illustrated in FIGS. 1 and 5A, one of ordinary skill in the art will recognize that the spacer 59 can be used in conjunction with other dual bracketed probes, such as, but not limited to, those with distal configurations as illustrated in FIGS. 4A through 4E. The spacer 59 can be provided in a kit that comprises at least the probes 1, 10, cables 31, 310, and optionally an energy source. In one aspect, more than one spacer 59 can be included in the kit. Different sized spacers having variously spaced bores 69, 690 could be included in the kit, depending on the desired ablation treatments.

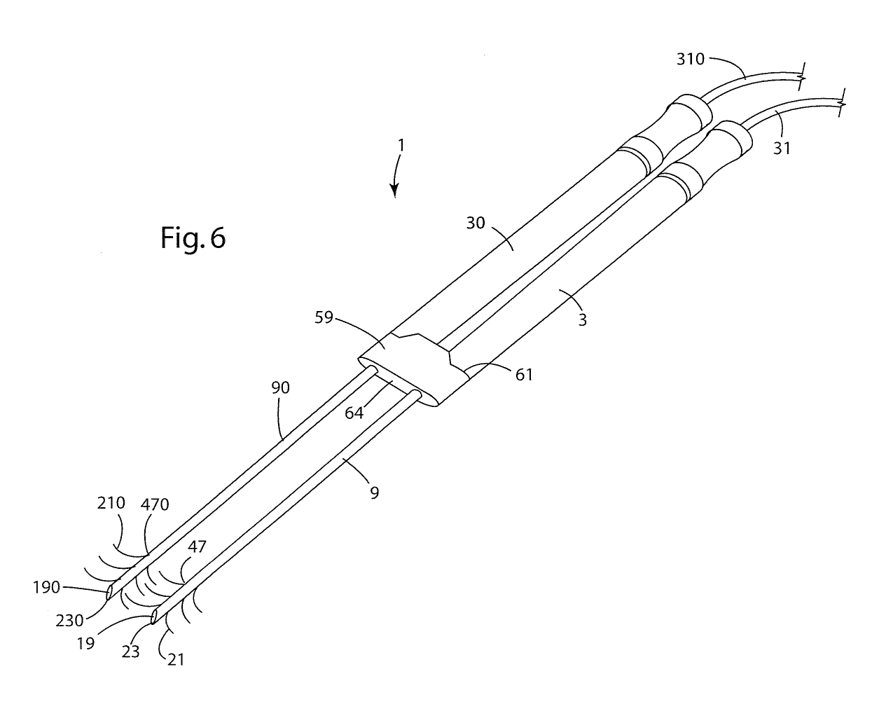

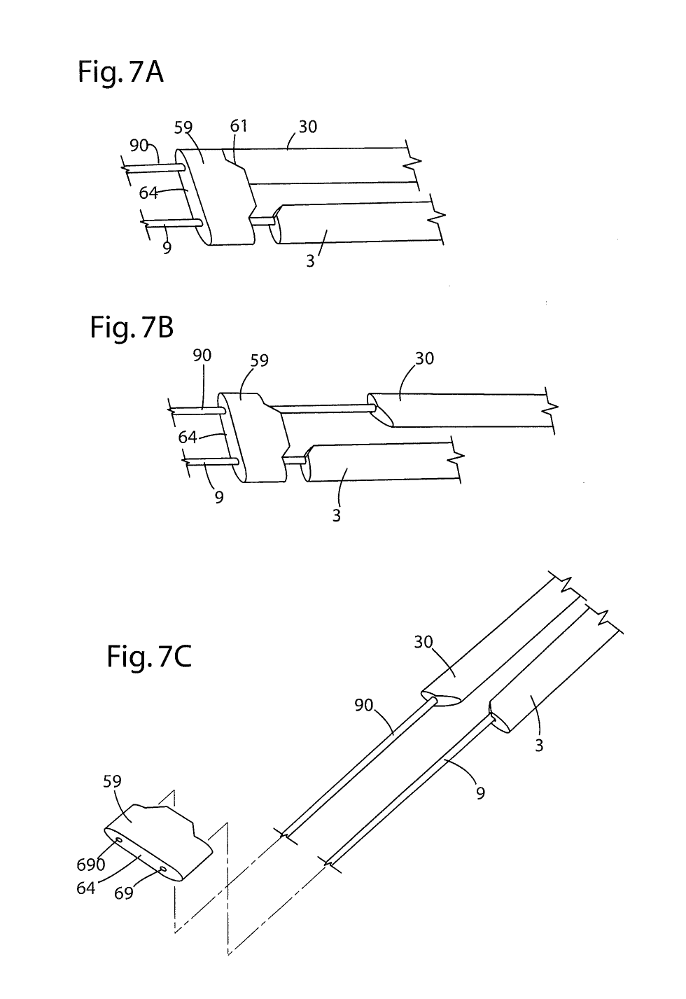

Referring to FIGS. 6 through 7C, another embodiment of an energy delivery probe 1 with a pre-assembled locking spacer 59 is described herein. In the pre-assembled configuration, a portion of the outer surface of the spacer 59 can be joined to the distal end 11 of the handle 3 along mating line 61. Particularly, the proximal end 95 of the spacer 59 can be joined to the handles 3, 30. The outer surface of the spacer 59 and the outer surfaces of the handles 3, 30 can be designed such that they form a moveable lock and key or tongue and groove fit. Although the spacer 59 illustrated in FIGS. 6 through 7C is shown in a pre-assembled configuration in one particular embodiment, one of ordinary skill in the art will recognize that the spacer 59 can be pre-assembled with any of the probe embodiments described herein.

This probe spacer 59 is advantageous because, as illustrated in FIGS. 7A through 7C, the position of one or both of the handles 3, 30, which are coupled to the trocars 9, 90 can be adjusted together or separately before or after insertion and use in a patient body, as needed. As illustrated in FIG. 7A, the first handle 3 and trocar 9 can be slideably moved proximally from the spacer 59, while the second handle 30 and trocar 90 remain stationary. The second handle 30 and trocar 90 can be separately slidably proximally moved, as illustrated in FIG. 7B. As illustrated in FIG. 7C, both handles 3, 30 and trocars 9, 90 can be completely removed from the spacer 59. Subsequently, one or both of the handles 3, 30 and trocars 9, 90 can be reinserted and repositioned through the bores 69, 690 of the spacer 59 for further use, if desired.

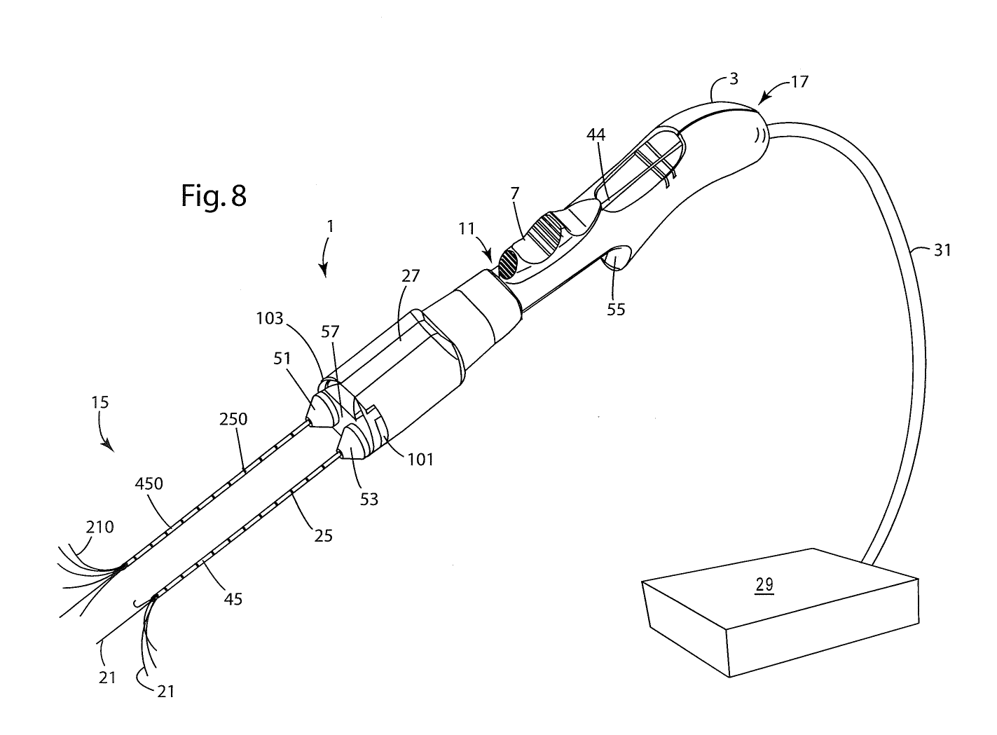

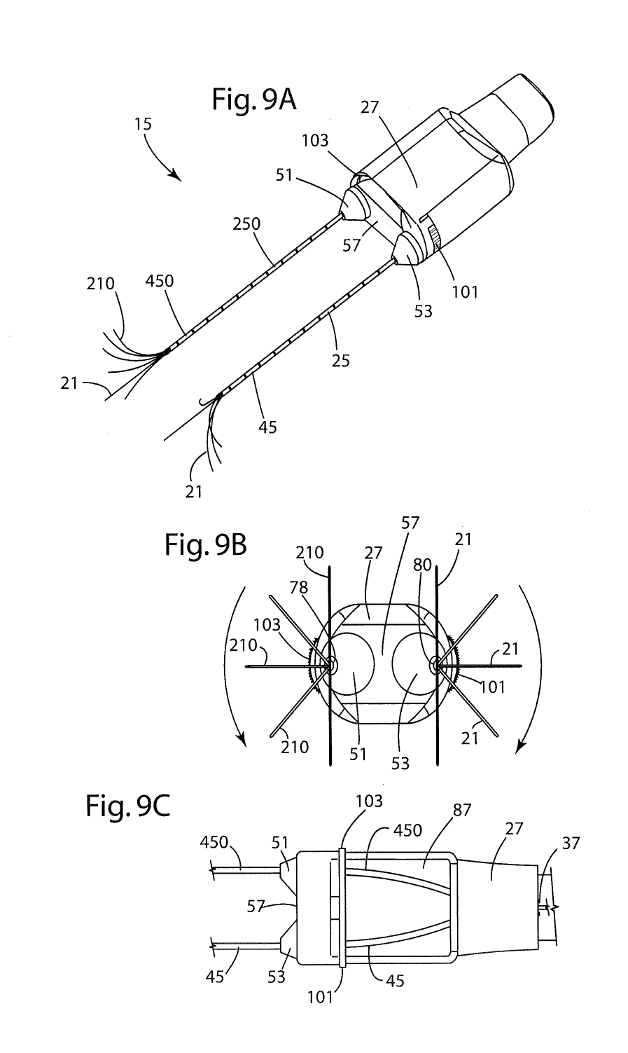

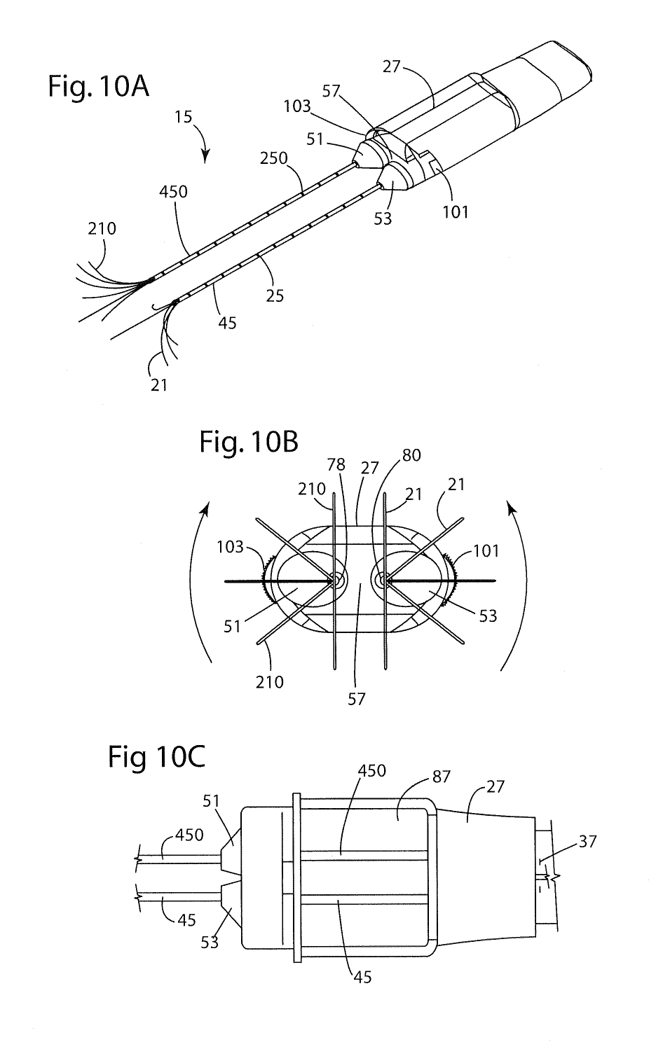

Referring to FIGS. 8 through 10C, another embodiment of the probe 1 is illustrated. This probe 1 is similar to the probes described above and illustrated in FIGS. 1 through 5A. In this embodiment, the handle 3 can be similar or identical to that of the StarBurst® XL probe (AngioDynamics, Inc., Latham, N.Y.). The probe 1 comprises a probe body. The body comprises a handle 3 that has a proximal end 17, a distal end 11, a slide member 7, a slot 44, and a grip 55. The probe body further comprises a cannula 27. The proximal end of the cannula 27 is permanently attached to the distal end 11 of the handle 3. The cannula 27 can be made of any suitable material, such as, but not limited to, ABS plastic or other similar plastics, such as PEEK. The cannula 27 has a proximal end and a distal end, an outer surface, a front face 57, and a cavity 87. The cannula 27 can be between about 9 and 11 cm in length, between about 3 cm and 5 cm in width, and about 1 cm and 3 cm in thickness, although one of ordinary skill in the art will recognize that other dimensions can be contemplated. At least a portion of trocars 9, 90 can be positioned within at least a portion of the cavity 87 of the cannula 27, as illustrated in FIGS. 9C and 10C. A portion of the electrodes 9, 90 extend distally from the cavity 87 of the cannula 27.

The cannula 27 can further comprise a first trocar or electrode holder 51 and a second trocar or electrode holder 53. Each of the trocar holders 51, 53 can be positioned next to each other within a portion of the front face 57 of the cannula 27 along a horizontal axis. Each trocar holder 51, 53 extends distally from the front face 57 of the cannula 27. The trocar holders 51, 53 and the trocars 9, 90 are positioned at a first position parallel to each other. As illustrated in FIGS. 8 and 9A, this first position can be a position in which the electrodes 9, 90 are positioned a maximum, parallel distance relative to each other.

Referring to FIG. 9B, each trocar holder 51, 53 has a front surface area that is divisible between a first portion and a second portion. The first and second portions are substantially equal in size and are divided by a horizontal axis. Each of the trocar holders 51, 53 has an opening 78, 80 that is positioned in the front surface of each of the trocar holders 51, 53 along an outer edge of the horizontal axis that extends across the face of the trocar holders 51, 53. A portion of each of the trocars 9, 90 extends distally through the openings 78, 80 of the trocar holders 51, 53.

Referring to FIGS. 8 through 10B, the cannula 27 further comprises a means for adjusting the position or the distance between the first trocar and the second trocar. Particularly, the means for adjusting can comprise a first finger-actuatable rotator 101 and a second finger-actuatable rotator 103. The means for adjusting is operatively coupled to the first trocar 9 and the second trocar 90. The first and second rotators 101, 103 are positioned within a portion of the cavity 87 of the cannula 27 and are capable of being manually rotated. Each of the rotators 101, 103 can have a ridged outer surface to provide traction for manual actuation of the rotators 101, 103. The rotators 101, 103 can be positioned such that the outer ridged surfaces extend beyond the outer surface of the cannula 27. Each rotator 101, 103 is actuatable along a first 180 degree arc and a second 180 degree arc, as indicated by the arrows in FIGS. 9B and 10B. These 180 degree arcs extend along a vertical axis that is substantially perpendicular to the horizontal axis of the trocar holders 51, 53.

A portion of each of the rotators 101, 103 is operatively coupled to a portion of each of a first gear and a second gear (not shown). The first gear and second gear are positioned within the cavity 87 of the cannula 27 at the distal end of the cannula 27. A portion of each of the first gear and the second gear is also operatively coupled to a portion of each of the trocars 9, 90 through a hole that is defined within each gear. As the first and second rotators 101, 103 are simultaneously actuated along the first and second 180 degree arcs that lie along the vertical axis, this causes the first and second gears to rotate. This in turn, causes the first and second trocar holders 51, 53 along with the first and second trocars 9, 90 to be simultaneously rotated along third and fourth mirrored opposite 180 degree arcs at the same rate of speed, but in opposite directions relative to each other. The third and fourth mirrored opposite 180 degree arcs are positioned such that a linear extension between the outermost points of the third and fourth 180 degree arcs is parallel to the horizontal axis. As the gears rotate, the trocars 9, 90 move freely within the holes of the gears. This rotation feature allows a user to adjust the position of the trocars 9, 90, depending on the size of the desired ablation, but yet maintain the trocars 9, 90 in a parallel position relative to each other before insertion, during treatment, and during withdrawal of the probe from a patient. This probe design also allows for single stage deployment of the dual bracketed energy delivery probe 1 for IRE or RF ablations, instead of using successive single probe devices or multiple probe devices at one time, as are currently used. The trocars 9, 90 are adapted to be adjustable between a first position in which they are positioned a maximum distance from each other of from between about 3 cm and about 5 cm, as illustrated in FIG. 8 through 9C, to a second position in which the trocars 9, 90 are positioned a distance that is less than the maximum distance from each other. In one aspect, the first position and the second position define a physical range of motion of the trocars 9, 90. The first trocar 9 and the second trocar 90 remain parallel to each other throughout the complete range of motion.

Referring to FIGS. 10A through 10C, the trocars 9, 90 can be positioned a minimum distance from each other of between about 0.5 cm and about 1 cm. Throughout the complete range of adjustment between a position of maximal spacing between the trocars and a position of minimum spacing between the trocars 9, 90, the trocars 9, 90 can be rotated such that they continuously remain parallel relative to each other throughout a complete range of adjustment. Any of the distal array 21 configurations illustrated in FIGS. 4A through 4E could be used in the probe 1 illustrated in FIGS. 8 through 100.

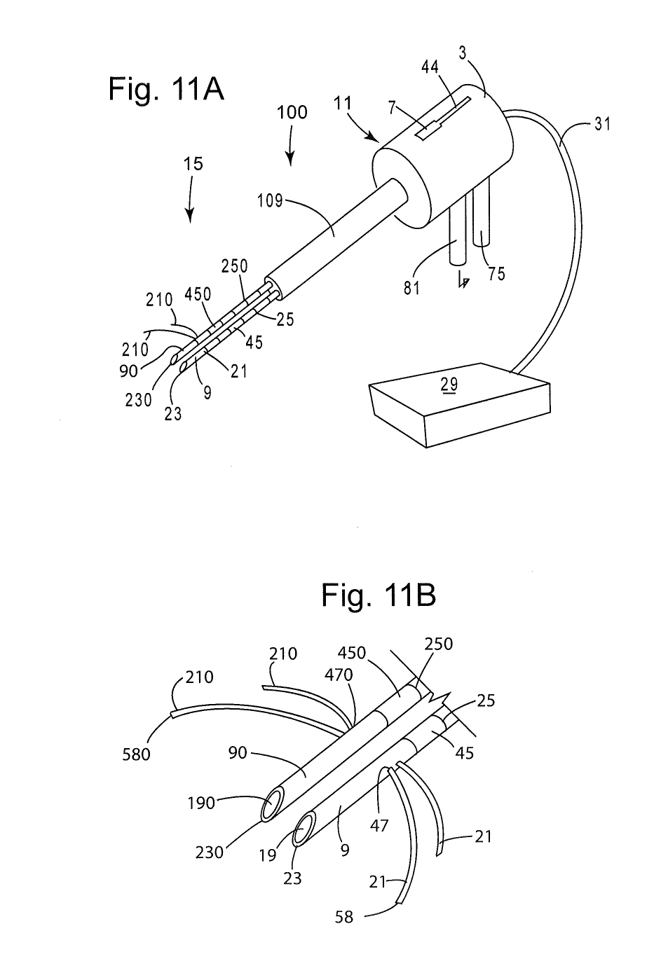

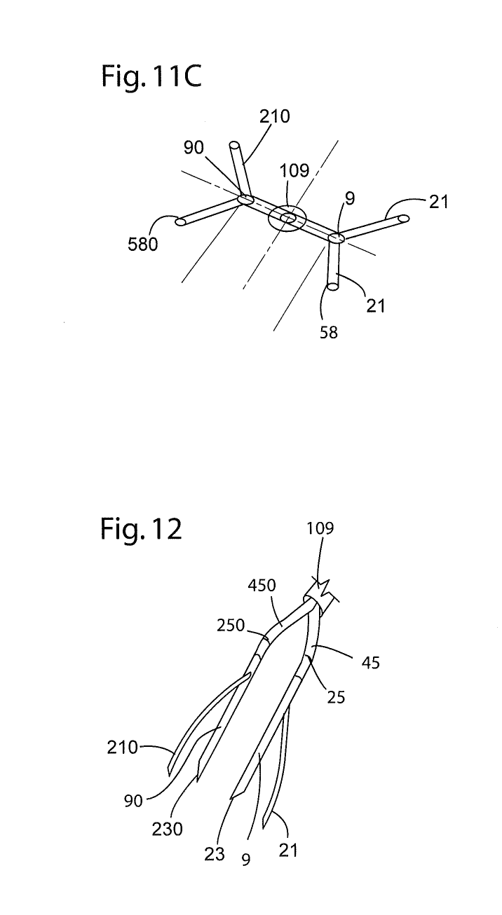

Referring to FIGS. 11A through 12, a different partial embodiment of the energy delivery probe 1 is illustrated. This device is a laparoscopic surgical device 100. This device 100 comprises a proximal end 17, a distal end 15, trocars 9, 90, two or more arrays 21, and a probe body. The probe body comprises a control handle 3 at the proximal end 17 and laparoscopic catheter 109. The device 100 is connected to an energy source, such as an RF energy source. Such RF energy source can be, but is not limited to, the AngioDynamics® RITA® 1500X generator. The distal end 11 of the handle 3 is attached to the proximal end of the laparoscopic catheter 109. In one aspect, the catheter 109 can be about 10 mm in diameter. The trocars 9, 90 can be positioned within a portion of the handle 3 and extend from the handle 3 through the catheter 109 distally from the catheter 109. The trocars 9, 90 are permanently positioned substantially parallel relative to each other along at least a portion of the longitudinal length of the trocars 9, 90.

Each of the trocars 9, 90 further comprises a distal tip 23 capable of piercing tissue and a hollow lumen through which a plurality of electrode arrays 21, 210 can be deployed along a radius of curvature into the tissue through openings 47. The probe 100 can comprise between about 2 and about 4 electrodes, although one of ordinary skill in the art will recognize that any suitable number of electrode arrays 21, 210 can be used. The trocars 9, 90 can be spaced apart approximately 1 cm. The trocars 9, 90 can be coaxially surrounded by an insulative sleeve 45, 450 similar to the embodiments described above. As illustrated in FIGS. 11A and 11B, the insulation sleeves 45, 450 coaxially surround each trocar 9, 90 for at least a partial length of the trocars 9, 90, as described above. The insulation sleeves 45, 450 can be approximately 0.006 inches in thickness. A portion of the insulation sleeves 45, 450 are operatively coupled to a finger-actuatable slide member 7.

The slide member 7 is capable of being actuated in either a proximal or distal direction along the longitudinal axis of the probe device 100. To retract the insulative sleeve 45, the slide member 7 can be manually proximally actuated. To advance the insulative sleeve 45, the slide member can be manually distally actuated. Handle 75 and trigger 81 can be coupled to a portion of the handle 3 opposite the slide member 7. Handle 75 is stationary and can be used as a grip. Trigger 81 is proximally slideably actuatable along a surface of the handle 3 along the direction of the arrow, as illustrated, and is operatively connected to the electrode arrays 21. Trigger 81 can be proximally actuated by a user in order to deploy arrays 21, 210 laterally from the trocars 9, 90.

In the embodiments illustrated in FIGS. 11A through 12, unlike the embodiments described above, the electrode arrays 21, 210 are not surrounded by an insulation sleeve 93. The electrode arrays 21, 2210 are capable of operating in a monopolar or a bipolar manner. During use, after the arrays 21 are deployed, the first trocar 9 and accompanying arrays 21 have a positive charge. The second trocar 90 and accompanying arrays 210 have a negative charge. The opposite polarities of these two sets of electrodes obviate the need to have an insulation sleeve positioned around any portion of the arrays 21, 210. This bare electrode array design is advantageous because it eliminates the chance that added insulation, particularly surrounding the curved portion of the arrays 21, 210, could become damaged during use.

FIG. 11B illustrates an enlarged distal end view of the laparoscopic device 100 of FIG. 11A. The electrode array configuration in this embodiment is useful for the treatment of larger tissue areas and/or for ensuring that a large enough ablation zone is created that is thick enough to close significant arteries. In this configuration, the electrode arrays 21, 210 extend outwardly from openings 47 to the sides of the device 100 such that the distance from tip 58 to tip 580 is approximately 3 cm.

FIG. 11C illustrates a front end view of the probe 100 illustrated in FIGS. 11A and 11B. This electrode configuration allows for an alternative ablation zone. FIG. 12 illustrates yet another embodiment of a distal array 21, 210 configuration of the laparoscopic probe 100. In this embodiment, the spacing between the trocars 9, 90 can transition from a first parallel position to a second parallel position distally of the catheter 109 along a longitudinal length of the trocars 9, 90. In the first position, the trocars are spaced a first parallel distance relative to each other. In the second position, the trocars are spaced a second, greater parallel distance relative to each other. When the arrays 21, 210 are completely deployed from the trocars 9, 90 into tissue along a radius of curvature the diameter between the tips 58, 580 of the outermost arrays 21, 210 is about 3 cm. This configuration provides for a substantially linear ablation zone.

One method of percutaneous insertion and use of the probe 1, illustrated in FIGS. 1 through 2B, for RF ablations or IRE ablations to treat a target tissue region is described and illustrated herein. The target tissue region can be a tissue or tumor that can be located in any of the following organs or tissue types: lung, liver, pancreas, breast, prostate, bone, stomach, kidney, spleen, uterus, brain, head, neck, colon, vascular, adipose, lymph, ovarian, eye, ear, bladder, skin, or any other desired mammalian target tissue area of a patient's body. The target tissue can comprise any one of the following tissue conditions within an organ or body tissue: benign prostate hyperplasia (BPH), uterine fibroids, malignant tissue, cancerous tissue, tumorous tissue, and benign tissue.

This method involves identifying a target tissue region having a first side and a second side, which sides are opposite from each other. An incision in a patient's skin can be optionally created. An ablation device can be provided, such as that described above and illustrated in FIGS. 1 through 2B having at least a first trocar 9 and a second trocar 90 and a plurality of electrode arrays 21. The first and second trocars 9, 90 are inserted into the target tissue such that the first trocar 9 and the second trocar 90 remain substantially parallel. This method further comprises positioning the first trocar 9 on the first side of the target tissue and the second trocar 90 on the second side of the target tissue. A plurality of electrode arrays 21 is deployed into the tissue from the trocars 9, 90. The method can further comprise actuating a slide member 7 to which the arrays 21, 210 are coupled such that the arrays 21, 210 can become fully deployed into the target tissue. During insertion, treatment, and withdrawal of the probe 1, the electrodes 9, 90 remain substantially parallel to each other. The method further involves delivering energy from an energy source 29 through the plurality of arrays 21 to a target tissue in order to ablate the target tissue, thereby forming a first ablation zone. The ablation zone can be defined as the radiologically identifiable region in which an ablation effect was directly induced. The ablation zone can extend between any point on the first side of the target tissue and any point on the second side of the target tissue.

Alternatively, the electrode arrays 21 may be positioned in a retracted state within the trocars 9, 90, as illustrated in FIGS. 3A through 3C, during the delivery of energy to the target tissue, and the method may further include delivering energy to the target tissue through the trocars 9, 90. In this aspect, the trocars can function like electrodes. In any of the methods described herein, the energy delivered to the target tissue can be radiofrequency energy. Alternatively, the energy delivered can be electrical energy in the form of electrical pulses that can be sufficient to cause non-thermal irreversible electroporation of the target tissue.

After a first ablation is completed, as described above, the method can further involve retracting the plurality of arrays 21, 210 from the target tissue into a portion of the trocars 9, 90, withdrawing the trocars 9, 90 from the target tissue, and optionally repeating the ablation procedure described above at the same or a different target tissue site.

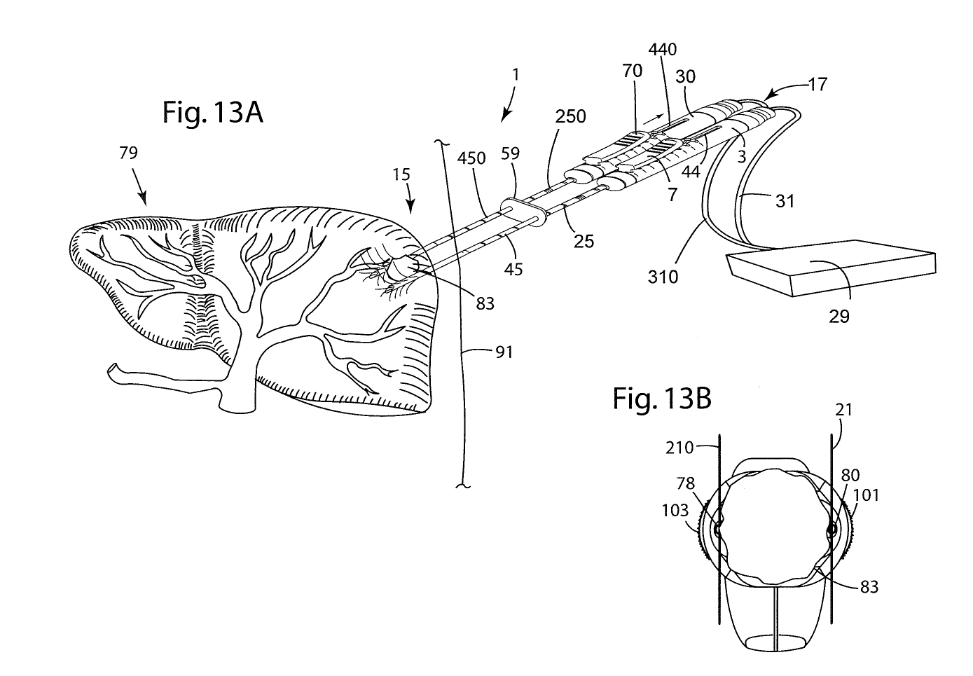

Referring to FIGS. 13A and 13B, one method of percutaneous insertion and use of the probe 1, also illustrated in FIG. 5A, for RF ablations or IRE ablations to percutaneously treat a target tissue region is described and illustrated herein. The target tissue region can be a tumor. This method is identical to the method described above, but also includes positioning a portion of a spacer 59 adjacent to a patient's skin after the target tissue has been identified, and an appropriate probe 1 has been provided. The distal end of the spacer 59 is placed against a patient's skin. The method further comprises inserting a first trocar 9 through a portion of the spacer 59. The trocar 9 can be inserted through a first bore 69 or a second bore 690 of the spacer 59. The method further involves positioning the first electrode 9 in or near the first side of the target tissue; inserting a second electrode 90 through a portion of the spacer 59, such as the first bore 69 or the second bore 690; positioning the second electrode 90 in or near the second side of the target tissue such that the first electrode 9 and the second electrode 90 remain substantially parallel; and adjusting the spacer 59 along the longitudinal length of the trocars 9, 90 to a desired position. The step can further comprise proximally sliding the spacer 59 along an outer surface of the longitudinal length of the trocars 9, 90 toward the probe bodies, and rotating the probes 1, 10 until they can be locked into place. Once locked into place, the locking mechanism in the spacer 59 can hold both the first trocar 9 and the second trocar 90 parallel to each other and at the same depth within the target tissue such that the target tissue is bracketed or surrounded throughout the entire ablation procedure.

The method further comprises deploying a plurality of electrode arrays 21, 210 into the target tissue; and delivering energy from an energy source 29 through the plurality of arrays 21, 210 to a target tissue in order to ablate the target tissue, thereby forming a first ablation zone. Alternatively, the electrode arrays 21 may remain in a retracted state within the trocars 9, 90, and the method may include delivering energy to the target tissue through the trocars 9, 90. The trocars 9, 90 can function like electrodes. The remaining steps of this method are identical to those described above. During insertion, treatment, and withdrawal of the probe 1, the trocars 9, 90 remain substantially parallel to each other.

In one aspect, after a first ablation is completed, the method can further involve retracting the plurality of arrays 21, 210 from the target tissue, withdrawing the first trocar 9 or the second trocar 90 from the spacer 59, adjusting the position of the spacer 59, reinserting the first trocar 9 or the second trocar 90 through a portion of the spacer 59, such as the first bore 69 or second bore 690, deploying a plurality of electrode arrays 21, 210 into the target tissue, and delivering energy from an energy source 29 through the plurality of arrays 21, 210 to the target tissue to ablate the target tissue, thereby forming a second ablation zone. In one aspect, although not illustrated, the first ablation zone and the second ablation zone can overlap in size. Any variety of different positions may be utilized to create a desired ablation geometry for selected tissue masses of different geometries and sizes.

This ablation procedure can be repeated multiple times to achieve a desired ablation zone(s). The method of use of any of the probe assemblies described herein presents a substantial advantage over conventional RF and IRE ablation methods. This probe design and method is advantageous because it allows for overlapping ablations without requiring the insertion of both electrodes at the same time.

The above method of use described for the unassembled spacer 59 used in conjunction with the probes 1, 10 can also be used with the assembled spacer 59 and probes 1, 10 illustrated in FIGS. 6 through 7C. This method is identical to the methods described above, except after the step of inserting the trocars 9, 90, the spacer 59 may be adjusted along the length of the electrodes 9, 90. After a first ablation is completed, the method can further comprise adjusting the position of the spacer 59 against the skin in relation to the tissue, as described above and performing one or more additional ablation procedures.

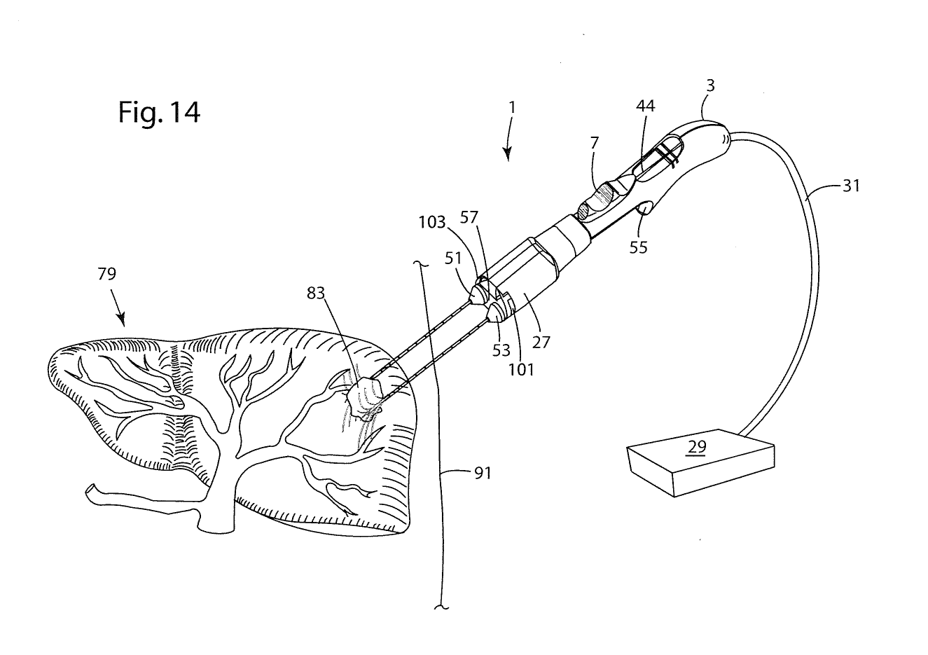

Referring to FIG. 14, another method of percutaneous insertion and use of the energy delivery probe 1 to percutaneously treat a target tissue region is described and illustrated herein. This method is identical to the methods described above, except this method comprises providing an ablation device illustrated in FIGS. 9A through 10C having a first electrode 9 and a second electrode 90 that are spaced in a first parallel position to each other. During insertion, treatment, and withdrawal of the probe 1, the electrodes 9, 90 remain substantially parallel to each other.

In this method, before inserting the probe 1 into the target tissue to perform a tissue ablation or after the probe 1 is withdrawn from the target tissue of a patient's body, the method can comprise adjusting the spacing between the first trocar 9 and the second trocar 90, reinserting the first trocar 9 and the second trocar 90, as described above, such that the first trocar 9 and the second trocar 90 remain substantially parallel to each other during insertion and use, and repeating the deployment and ablation steps, thereby forming a second ablation zone. In one aspect, although not illustrated, the first ablation zone and the second ablation zone can overlap in size.

In order to adjust the spacing of the first electrode 9 and the second electrode 90 relative to each other, this method can further involve actuating a means for adjusting the position of the trocars 9, 90 relative to each other by manually actuating at least one rotator 101, 103. As the rotators 101, 103 are manually actuated the trocars 9, 90 can be adjusted from a first position, wherein the first and second trocars are parallel to each other, to a second position wherein the trocars 9, 90 are parallel to each other. The first position of the trocars 9, 90 can be a position in which the trocars are spaced a maximum parallel distance relative to each other, and the second position can be a position in which the trocars 9, 90 are spaced a minimum parallel distance relative each other. The spacing between the trocars 9, 90 can be adjusted based on the size of the target tissue that is to be treated. In one aspect, the trocars 9, 90 can be spaced so that trocar 9 is positioned on a first side of the tumor and trocar 90 is positioned on the second side of the tumor so that the tumor can be positioned between the trocars on either side, as illustrated in FIG. 14.

During the methods described above, energy can be applied from the energy source or generator 29 between the electrodes 21, 210 in various patterns. Particularly, electrical pulses of various voltages can be applied to the target tissue. In one aspect, as illustrated in FIG. 15A, energy can be applied from arrays 1 to 6, 2 to 5, and 3 to 4. In another aspect, as illustrated in FIG. 15B, energy can be applied from electrodes 1 to 4, 2 to 5, 3 to 6, 2 to 6, 3 to 5, 4 to 2, and 1 to 5. Alternatively, as illustrated in FIG. 15C, energy can be delivered between 1 and 2, 1 and 3, and 2 and 3. Each of these ablation patterns illustrated in FIGS. 15A through 15C is capable of producing substantially similarly sized ablation zones.

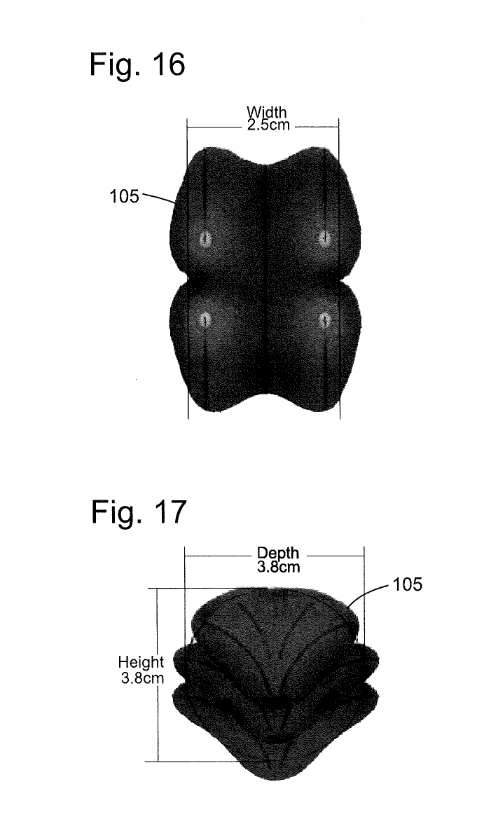

Software can be used to predict ablation zones using various probe configurations. As illustrated in FIGS. 16 and 17, plots outlining a predicted ablation zone 105 were obtained using the finite element method (“FEM”) COMSOL Multiphysics Modeling and Simulation software (Palo Alto, Calif.). In one aspect, as illustrated in FIG. 16, as viewed from the distal end of the trocars 9, 90, when the trocars are about 2 cm apart, a substantially rectangular ablation zone 105 that is approximately 2.5 cm wide was predicted. In one aspect, as illustrated in FIG. 17, the ablation zone 105 was predicted to be approximately 3.8 cm in depth by 3.8 cm in height.

### Example 1

IRE ablations were performed on 10 different pig liver tissues 107 using an energy delivery probe 1 as illustrated in FIG. 14. To perform the IRE ablation treatment, the probe 1 was percutaneously inserted into the pig liver tissue as described above, and 90 electric pulses of a 70 μsec pulse length were delivered per pair of electrodes 9, 90 at a voltage gradient of 1250 V/cm to each of the target pig liver tissues 107. Other suitable pulse parameters may be used. Voltage gradient (electric field) is a function of the distance between electrodes and electrode geometry, which will vary depending on the size of the tissue sample, tissue properties, and other factors. The amplitude of voltage pulses, duration of each pulse, total number of voltage pulses, and duration between consecutive pulses can be altered, depending on the desired ablation. IRE ablations, when carried out under certain parameters and operating conditions, can selectively spare certain tissues and structures present within the ablation volume. Non-limiting tissues that can be selectably spared by the pulsed electric field ablation include nervous, vascular structures, neural tubes, and ducts, as well as collagen-rich tissues.

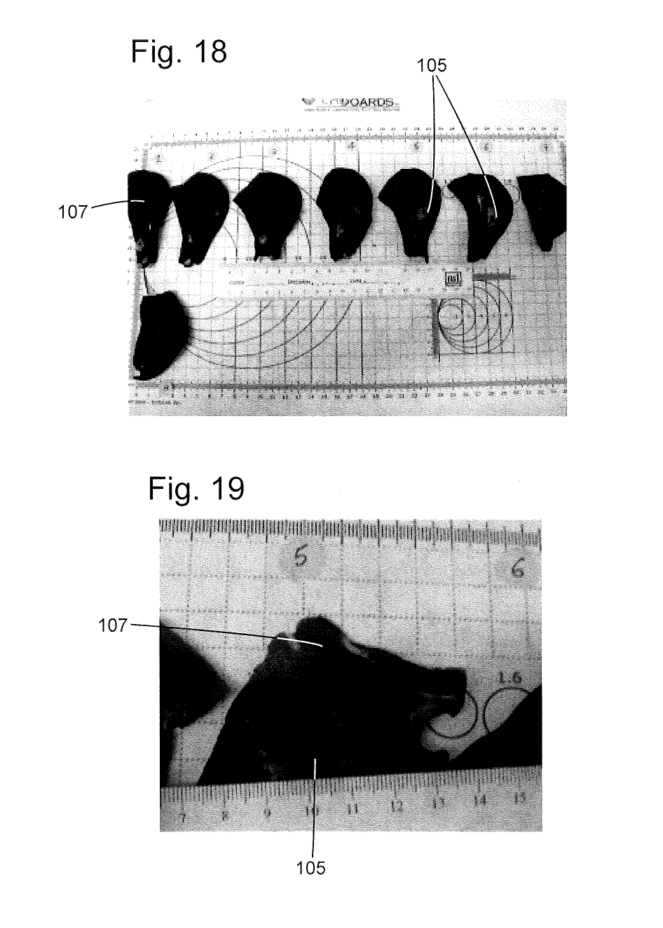

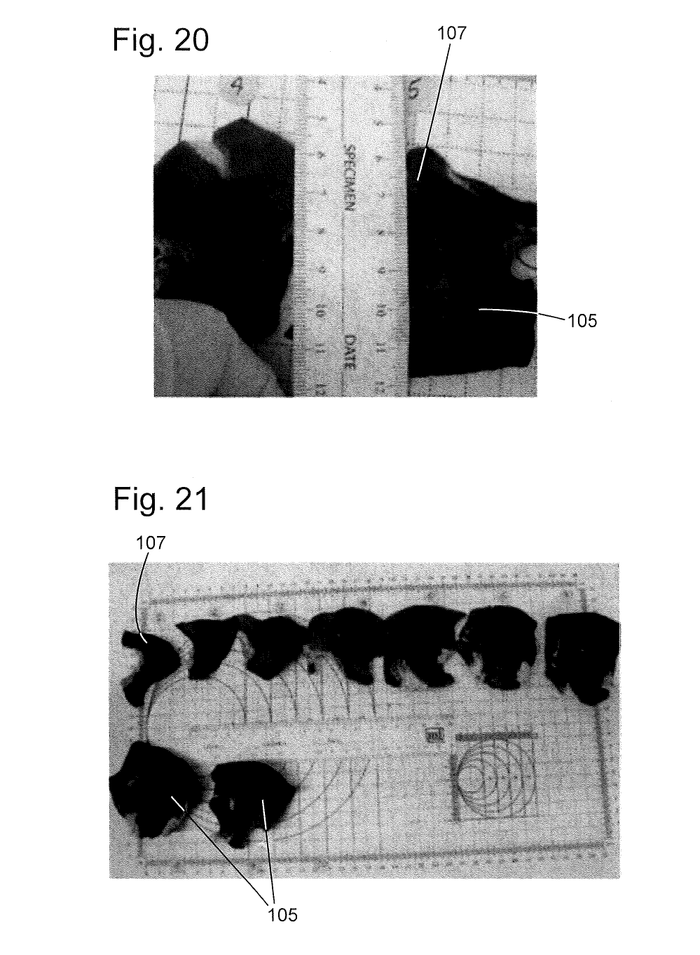

After the ablation procedure, the ablated liver tissues were removed from the animals. The liver tissue ablations were sliced perpendicularly to the electrodes 9, 90 into slices that were approximately 7 mm in thickness. Each pig liver tissue slice was then soaked in formalin for a minimum of 24 hours. The ablation zones 105 were measured, as illustrated in FIGS. 18 through 20. Each ablation zone 105 was approximately 5.6 cm in height, along the “Z” axis of a three-dimensional axis. The diameter of the ablation zone 105 was determined my multiplying 0.7 mm, or the thickness of each slice, by 8 slices. Liver tissue sections 1 and 9 were excluded due to the size of the ablation zones in these tissue samples. The COMSOL software predicted that the ablation zone 105 of the ablated tissue in these liver tissue samples 107 would be between about 3.8 cm to about 4 cm in the “Z” axis, when subtracting the minor peaks around the trocars 9, 90. The width of each ablation zone 105, as measured along the horizontal “X” and “Y” axes, was approximately 5 cm, as illustrated in FIG. 19. The COMSOL software predicted an ablation zone of approximately 2.5 cm in the “X” and “Y” axes. The ablation zone 105 along the vertical axis was approximately 3.8 cm, as illustrated in FIG. 20. This measurement was identical to the COMSOL ablation zone prediction of approximately 3.8 cm.

### Example 2

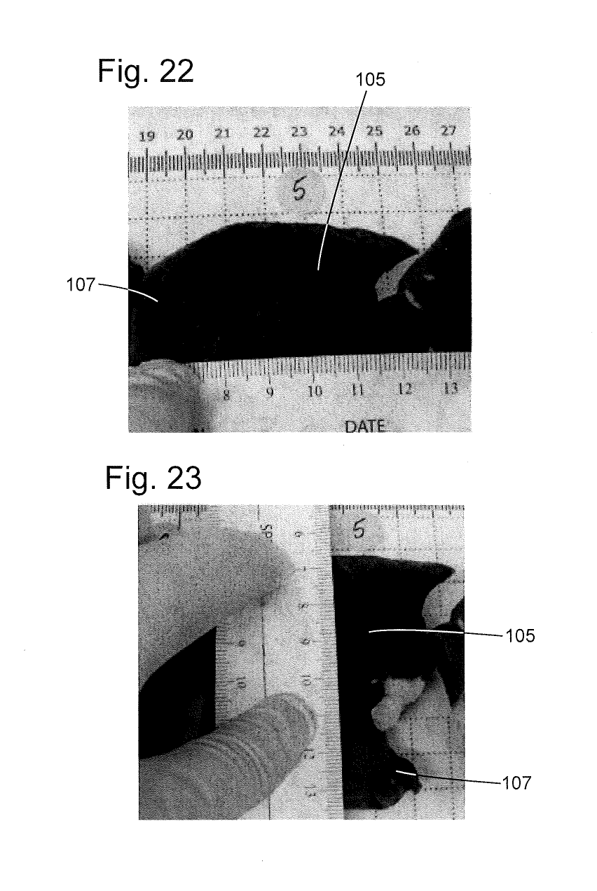

In this example, as illustrated in FIGS. 21 through 23, IRE ablations were performed on 9 different pig liver tissues 107 using an energy delivery probe 1 having a distal tip configuration as illustrated in FIG. 14. The IRE ablation procedure was repeated as described in Example 1. Each ablation zone 105 was approximately 5.6 cm in height, along a “Z” axis of a three-dimensional axis. The diameter of the ablation zone 105 was determined my multiplying 0.7 mm, or the thickness of each slice, by 7 slices. Liver tissue sections 1 and 9 were excluded due to the size of the ablation zones in these tissue samples. The COMSOL software predicted that the ablation zone 105 of the ablated tissue in these liver tissue samples 107 would be between about 3.8 cm to about 4 cm in the “Z” axis, when subtracting the minor peaks around the trocars 9, 90. The width of each ablation zone 105, as measured along the horizontal “X” and “Y” axes, was approximately 5 cm, as illustrated in FIG. 22. The COMSOL software predicted an ablation zone of approximately 2.5 cm in the “X” and “Y” axes. The ablation zone 105 along the vertical axis was approximately 4 cm, as illustrated in FIG. 23. This measurement was identical to the COMSOL ablation zone prediction of approximately 3.8 cm.

### Example 3

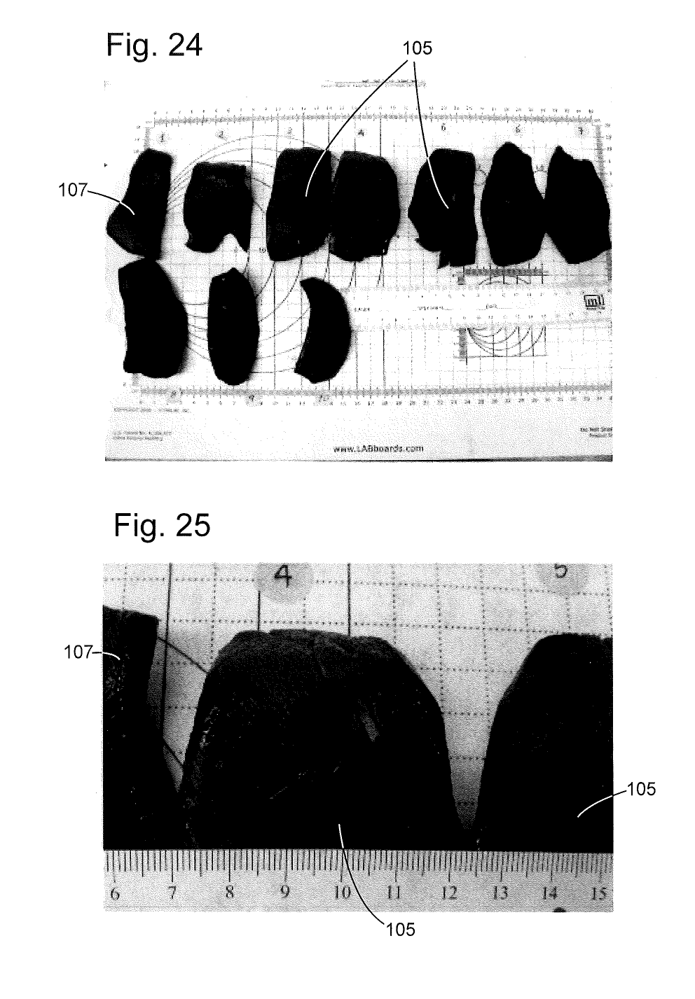

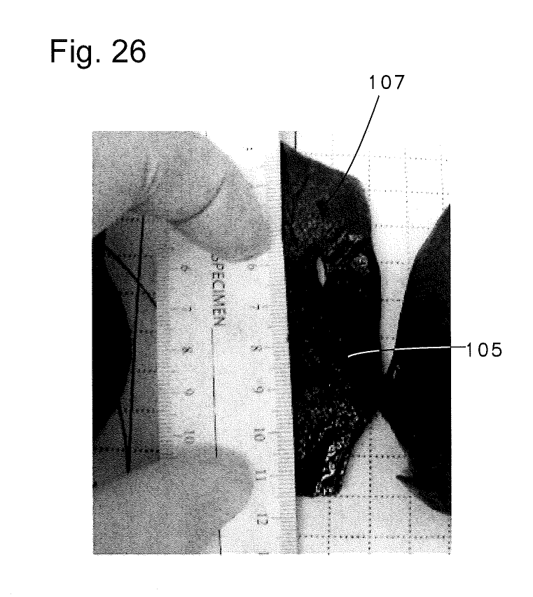

In this example, as illustrated in FIGS. 24 through 26, IRE ablations were performed on 10 different pig liver tissues 107 using an energy delivery probe 1 having a distal tip configuration as illustrated in FIG. 14. The procedure was repeated as described in Examples 1 and 2. Each ablation zone 105 was approximately 5.6 cm in height, along a “Z” axis of a three-dimensional axis. The diameter of the ablation zone 105 was determined my multiplying 0.7 mm, or the thickness of each slice, by 7 slices. Liver tissue sections 1, 9, and 10 were excluded due to the size of the ablation zones in these liver tissue samples 107. The COMSOL software predicted that the ablation zone 105 of the ablated tissue in these liver tissue samples 107 would be between about 3.8 cm to about 4 cm in the “Z” axis, when subtracting the minor peaks around the trocars 9, 90. The width of each ablation zone 105, as measured along the horizontal “X” and “Y” axes, was approximately 4 cm, as illustrated in FIG. 25. The COMSOL software predicted an ablation zone of approximately 2.5 cm along the “X” and “Y” axes. The ablation zone 105 along the vertical axis was approximately 4 cm, as illustrated in FIG. 26.

TABLE 1
Below is a table summarizing the results of the experimental
data from the above-described Examples.

| | COMSOL | | | |
|---|---|---|---|---|
| | Estimated ablation | | | |
| | size and shape (H × | | | |
| Example | W × D) at 2800 | Delivered | Ablation Size | |
| No. | volts | Voltage | (H × W × D) | Results |
| Ex. 1 | 3.8 × 2.5 × 3.8 | 2750 V | 5.6 × 5 × 3.8 | Complete |
| | Rectangular shape | | | ablation |
| Ex. 2 | 3.8 × 2.5 × 3.8 | 2520 V | 5 × 3 × 4 | Complete |
| | Rectangular shape | | | ablation |
| Ex. 3 | 3.8 × 2.5 × 3.8 | 2750 V | 5 × 4 × 4 | Complete |
| | Rectangular shape | | | ablation |
| Average | | | 5.2 × 4 × 3.9 | |
| Standard | | | .35 × 1 × .12 | |
| Deviation | | | | |

These IRE ablation methods, as disclosed in Examples 1 through 3, using the probes described herein can produce IRE ablation zones equal to or greater than about 2 cm in diameter. Particularly, the energy delivery probes 1 described herein can produce IRE ablation zones equal to or greater than about 3.5 cm in diameter. A variety of different geometric ablations for the ablation zone can be achieved, including, but not limited to oblong, circular, linear, spherical, semi-spherical, spheroid, triangular, semi-triangular, square, semi-square, rectangular, semi-rectangular, conical, semi-conical, quadrilateral, semi-quadrilateral, semi-quadrilateral, rhomboidal, semi-rhomboidal, trapezoidal, semi-trapezoidal, combinations of the preceding, geometries with non-planar sections or sides, free-form and the like.

A method for using the laparoscopic surgical probe 100 illustrated in FIGS. 11A through 12 is described herein. This device can be used as a bipolar resection device and can be used to assist in coagulation of tissue during intraoperative and laparoscopic surgical and resection procedures. This device can be used in laparoscopic resection procedures by employing RF energy to develop a plane of coagulative necrosis along an intended line of transection. The tissue can subsequently be divided with a scalpel through this zone of necrosis.

Typically, probe 100 will be used in conjunction with a suitable imaging system such as for example ultrasound, x-ray, MRI, or CT. In one aspect, the method of using this device involves identifying a target tissue, such as any of those described herein. The method further comprises providing an ablation device, such as that described above and illustrated in FIGS. 11A through 12 having at least a first trocar 9 and a second trocar 90, the first and the second trocar 9, 90 being parallel to each other, and a plurality of arrays 21, 210; and inserting the first and second trocars 9, 90 into the target tissue. The trocars 9, 90 help to stabilize the target tissue, such as a tumor. During insertion, treatment, and withdrawal of the probe 1, the trocars 9, 90 remain substantially parallel to each other. The catheter 109 is then inserted, typically via the abdominal wall, into an organ such as the liver 79. The trocars 9, 90 are moved into the organ guided by ultrasound, or any other available imaging technique until the desired location is reached. This method further involves deploying a plurality of electrode arrays 21, 210 into the target tissue. The step of deploying a plurality of arrays 21, 210 into the target tissue can further comprise actuating a trigger 81 to which the electrode arrays 21, 210 are coupled such that the electrode arrays 21, 210 can become fully deployed into the target tissue. The trigger 81 can be moved proximally to deploy the electrode arrays 21, 210 into the target tissue area to be treated.

The method further involves delivering energy from an energy source 29 through the plurality of electrode arrays 21, 210 to a target tissue in order to ablate the target tissue, thereby forming a first ablation zone. The energy delivered to the target tissue can be radiofrequency energy. When the RF energy is delivered to the target tissue, the target tissue surrounding a tumor is embolized, thereby cutting off a tumor's blood supply. Once the target tissue is treated, it can be resected.

After a first ablation is completed, as described above, the method can further involve retracting the plurality of electrode arrays 21, 210 from the target tissue into a portion of the trocars 9, 90; withdrawing the laparoscopic device 100 from the tissue and optionally repeating the ablation procedure described above. The method of using this device is advantageous because the parallel trocars 9, 90 can be used to create a coagulation resection line using the same probe that is used for tumor ablation.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. The words “including” and “having,” as used herein including the claims, shall have the same meaning as the word “comprising.” Those familiar with the art can recognize other equivalents to the specific embodiments described herein, which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g., each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

Therefore, it is to be understood that the embodiments of the invention are not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions can be provided by alternative embodiments without departing from the scope of the appended claims. In this regard, for example, different combinations of elements and/or functions than those explicitly described above are also contemplated as can be set forth in some of the appended claims.

This completes the description of the selected embodiments of the invention. Those skilled in the art can recognize other equivalents to the specific embodiments described herein which equivalents are intended to be encompassed by the claims attached hereto.

What is claimed is:

1. A probe system for ablating tissue comprising:
a first trocar comprising a first proximal end and a first distal end, wherein the first distal end comprises a first distal tip configured to pierce tissue and a first plurality of electrodes configured to be deployed away from the first trocar;
a second trocar comprising a second proximal end and a second distal end, wherein the second distal end comprises a second distal tip configured to pierce tissue and a second plurality of electrodes configured to be deployed away from the second trocar; and
a generator electrically coupled to the first plurality of electrodes and the second plurality of electrodes;
wherein a spacer connected to the first trocar and the second trocar maintains a fixed distance between the first trocar and the second trocar.

2. The probe system of claim 1, wherein the spacer maintains parallel alignment of the first trocar and the second trocar.

3. The probe system of claim 2, wherein the spacer is configured to allow the first trocar to slide through the spacer independent of the second trocar.

4. The probe system of claim 1, wherein at least one of the first plurality of electrodes deploys radially away from the first trocar.

5. The probe system of claim 1, wherein at least one of the second plurality of electrodes deploys radially away from the second trocar.

6. The probe system of claim 1, wherein the generator is configured to send an RF signal to the first and second plurality of electrodes.

7. The probe system of claim 1, wherein the generator is configured to send an electric signal to the first and second plurality of electrodes sufficient to irreversibly electroporate target tissue.

8. The probe system of claim 1, wherein at least one of the first plurality of electrodes is a needle electrode.

9. The probe system of claim 8, wherein the at least one electrode is coaxially surrounded by an insulation member.

10. The probe system of claim 9, wherein the insulation member is configured to adjust the exposure of the at least one electrode.

11. The probe system of claim 1, wherein a first actuating member is configured to deploy the first plurality of electrodes.

12. The probe system of claim 11, wherein a first set of markers are disposed on the probe system for indicating a deployment state of the first plurality of electrodes.

13. The probe system of claim 11, wherein a second actuating member is configured to deploy the second plurality of electrodes.

14. The probe system of claim 13, wherein a second set of markers are disposed on the probe system for indicating a deployment state of the second plurality of electrodes.

15. The probe system of claim 13, wherein the first set of markers and the second set of markers correspond to common deployment states of the first plurality of electrodes and the second plurality of electrodes.

16. The probe system of claim 1, wherein the generator is configured to apply an electrical signal between a first electrode and a second electrode of the first plurality of electrodes sufficient to irreversibly electroporate tissue between the first electrode and the second electrode.

17. The probe system of claim 1, wherein the generator is configured to apply an electrical signal between a first electrode of the first plurality of electrodes and a second electrode of the second plurality of electrodes sufficient to irreversibly electroporate tissue between the first electrode and the second electrode.

18. The probe system of claim 1, wherein the spacer is configured to adjust the fixed distance between the first trocar and the second trocar.

19. The probe system of claim 18, wherein the spacer comprises a locking mechanism for locking the fixed distance.

20. The probe system of claim 1, wherein the probe comprises a locking mechanism for locking an exposure of the first trocar in relative to an insulation member.
